(19) United States
(12) Patent Application Publication
(10) Pub. No.: US 2008/0103151 A9
(43) Pub. Date: May 1, 2008

(54) Heterocyclic compounds and their uses as therapeutic agents

(75) Inventors: Mikhail Chafeev, Burnaby (CA); Sultan Chowdhury, Scarborough (CA); Robert Fraser, North Vancouver (CA); Jianmin Fu, Coquitlam (CA); Rajender Kamboj, Burnaby (CA); Shifeng Liu, Port Coquitlam (CA); Vandna Raina, Burnaby (CA); Mehran Seid Bagherzadeh, North Vancouver (CA); Jianyu Sun, Richmond (CA); Serguei Sviridov, Burnaby (CA)

Correspondence Address: SEED INTELLECTUAL PROPERTY LAW GROUP PLLC, 701 FIFTH AVE, SUITE 5400, SEATTLE, WA, 98104, US

(73) Assignee: Xenon Pharmaceuticals Inc., Burnaby (CA)

(21) Appl. No.: 11/407,859

(22) Filed: Apr. 20, 2006

Related U.S. Application Data
(60) Provisional application No. 60/673,423, filed on Apr. 20, 2005.

(51) Int. Cl. A61K 31/519; A61K 31/498; A61K 31/4745; A61K 31/4162; A61K 31/407; C07D 487/02; C07D 498/02

(52) U.S. Cl. 514/248; 514/265.1; 514/300; 514/406; 514/412; 544/280; 544/350; 546/113; 548/360.5; 548/453

(57) ABSTRACT

This invention is directed to heterocyclic compounds that are useful for the treatment and/or prevention of sodium channel-mediated diseases or conditions, such as pain. Pharmaceutical compositions comprising the compounds and methods of using the compounds are also disclosed.

### CROSS-REFERENCE(S) TO RELATED APPLICATION(S)

This application claims the benefit under 37 U.S.C. §119(e) of U.S. Provisional Patent Application No. 60/673,423 filed Apr. 20, 2005, which application is incorporated herein by reference in its entirety.

### FIELD OF THE INVENTION

The present invention is directed to heterocyclic compounds. In particular, this invention is directed to heterocyclic compounds that are sodium channel blockers and are therefore useful in treating sodium channel-mediated diseases or conditions, such as pain, as well as other diseases and conditions associated with the mediation of sodium channels.

### BACKGROUND OF THE INVENTION

Voltage-gated sodium channels, transmembrane proteins that initiate action potentials in nerve, muscle and other electrically excitable cells, are a necessary component of normal sensation, emotions, thoughts and movements (Catterall, W. A., Nature (2001), Vol. 409:988-990). These channels consist of a highly processed alpha subunit that is associated with auxiliary beta subunits. The pore-forming alpha subunit is sufficient for channel function, but the kinetics and voltage dependence of channel gating are in part modified by the beta subunits (Goldin et al., Neuron (2000), Vol. 28:365-368). Each alpha-subunit contains four homologous domains, I to IV, each with six predicted transmembrane segments. The alpha-subunit of the sodium channel, forming the ion-conducting pore and containing the voltage sensors regulating sodium ion conduction has a relative molecular mass of 260,000. Electrophysiological recording, biochemical purification, and molecular cloning have identified ten different sodium channel alpha subunits and four beta subunits (Yu, F. H., et al, Sci STKE (2004), 253; and Yu, F. H., et al., Neurosci. (2003), 20:7577-85).

The hallmarks of sodium channels include rapid activation and inactivation when the voltage across the plasma membrane of an excitable cell is depolarized (voltage-dependent gating), and efficient and selective conduction of sodium ions through conducting pores intrinsic to the structure of the protein (Sato, C., et al., Nature (2001), 409:1047-1051). At negative or hyperpolarized membrane potentials, sodium channels are closed. Following membrane depolarization, sodium channels open rapidly and then inactivate. Channels only conduct currents in the open state and, once inactivated, have to return to the resting state, favoured by membrane hyperpolarization, before they can reopen. Different sodium channel subtypes vary in the voltage range over which they activate and inactivate as well as their activation and inactivation kinetics.

The sodium channel family of proteins has been extensively studied and shown to be involved in a number of vital body functions. Research in this area has identified variants of the alpha subunits that result in major changes in channel function and activities, which can ultimately lead to major pathophysiological conditions. Implicit with function, this family of proteins are considered prime points of therapeutic intervention. Naᵥ1.1 and Naᵥ1.2 are highly expressed in the brain (Raymond, C. K., et al., J. Biol. Chem. (2004), 279(44):46234-41) and are vital to normal brain function. In humans, mutations in Naᵥ1.1 and Naᵥ1.2 result in severe epileptic states and in some cases mental decline (Rhodes, T. H., et al., Proc. Natl. Acad. Sci. USA (2004), 101(30):11147-52; Kamiya, K., et al., J. Biol. Chem. (2004), 24(11):2690-8; Pereira, S., et al., Neurology (2004), 63(1):191-2). As such both channels have been considered as validated targets for the treatment of epilepsy (see PCT Published Patent Publication No. WO 01/38564).

Naᵥ1.3 is broadly expressed throughout the body (Raymond, C. K., et al., op. cit.). It has been demonstrated to have its expression upregulated in the dorsal horn sensory neurons of rats after nervous system injury (Hains, B. D., et al., J. Neurosc. (2003), 23(26):8881-92). Many experts in the field have considered Naᵥ1.3 as a suitable target for pain therapeutics (Lai, J., et al., Curr. Opin. Neurobiol. (2003), (3):291-72003; Wood, J. N., et al., J. Neurobiol. (2004), 61(1):55-71; Chung, J. M., et al., Novartis Found. Symp. (2004), 261:19-27; discussion 27-31, 47-54).

Naᵥ1.4 expression is essentially limited to muscle (Raymond, C. K., et al., op. cit.). Mutations in this gene have been shown to have profound effects on muscle function including paralysis, (Tamaoka A., Intern. Med. (2003), (9):769-70). Thus, this channel can be considered a target for the treatment of abnormal muscle contractility, spasm or paralysis.

The cardiac sodium channel, Naᵥ1.5, is expressed mainly in the heart ventricles and atria (Raymond, C. K., et al., op. cit.), and can be found in the sinovial node, ventricular node and possibly Purkinje cells. The rapid upstroke of the cardiac action potential and the rapid impulse conduction through cardiac tissue is due to the opening of Naᵥ1.5. As such, Naᵥ1.5 is central to the genesis of cardiac arrhythmias. Mutations in human Naᵥ1.5 result in multiple arrhythmic syndromes, including, for example, long QT3 (LQT3), Brugada syndrome (BS), an inherited cardiac conduction defect, sudden unexpected nocturnal death syndrome (SUNDS) and sudden infant death syndrome (SIDS) (Liu, H. et al., Am. J. Pharmacogenomics (2003), 3(3):173-9). Sodium channel blocker therapy has been used extensively in treating cardiac arrhythmias. The first antiarrhythmic drug, quinidine, discovered in 1914, is classified as a sodium channel blocker.

Naᵥ1.6 encodes an abundant, widely distributed voltage-gated sodium channel found throughout the central and peripheral nervous systems, clustered in the nodes of Ranvier of neural axons (Caldwell, J. H., et al., Proc. Natl. Acad. Sci. USA (2000), 97(10):5616-20). Although no mutations in humans have been detected, Naᵥ1.6 is thought to play a role in the manifestation of the symptoms associated with multiple sclerosis and has been considered as a target for the treatment of this disease (Craner, M. J., et al., Proc. Natl. Acad. Sci. USA (2004), 101(21):8168-73).

Naᵥ1.7 was first cloned from the pheochromocytoma PC12 cell line (Toledo-Aral, J. J., et al., Proc. Natl. Acad. Sci. USA (1997), 94:1527-1532). Its presence at high levels in the growth cones of small-diameter neurons suggested that it could play a role in the transmission of nociceptive information. Although this has been challenged by experts in the field as Naᵥ1.7 is also expressed in neuroendocrine cells associated with the autonomic system (Klugbauer, N., et al., EMBO J. (1995), 14(6):1084-90) and as such has been implicated in autonomic processes. The implicit role in autonomic functions was demonstrated with the generation of Naᵥ1.7 null mutants; deleting Naᵥ1.7 in all sensory and sympathetic neurons resulted in a lethal perinatal phenotype. (Nassar, et al., Proc. Natl. Acad. Sci. USA (2004), 101(34):12706-11.). In contrast, by deleting the Naᵥ1.7 expression in a subset of sensory neurons that are predominantly nociceptive, a role in pain mechanisms, was demonstrated (Nassar, et al., op. cit.). Further support for Naᵥ1.7 blockers active in a subset of neurons is supported by the finding that two human heritable pain conditions, primary erythermalgia and familial rectal pain, have been shown to map to Naᵥ1.7 (Yang, Y., et al., J. Med. Genet. (2004), 41(3):171-4).

The expression of Naᵥ1.8 is essentially restricted to the DRG (Raymond, C. K., et al., op. cit.). There are no identified human mutations for Naᵥ1.8. However, Naᵥ1.8-null mutant mice were viable, fertile and normal in appearance. A pronounced analgesia to noxious mechanical stimuli, small deficits in noxious thermoreception and delayed development of inflammatory hyperalgesia suggested to the researchers that Naᵥ1.8 plays a major role in pain signalling (Akopian, A. N., et al., Nat. Neurosci. (1999), 2(6):541-8). Blocking of this channel is widely accepted as a potential treatment for pain (Lai, J, et al., op. cit.; Wood, J. N., et al., op. cit.; Chung, J. M., et al., op. cit.). PCT Published Patent Application No. WO03/037274A2 describes pyrazole-amides and sulfonamides for the treatment of central or peripheral nervous system conditions, particularly pain and chronic pain by blocking sodium channels associated with the onset or recurrance of the indicated conditions. PCT Published Patent Application No. WO03/037890A2 describes piperidines for the treatment of central or peripheral nervous system conditions, particularly pain and chronic pain by blocking sodium channels associated with the onset or recurrence of the indicated conditions. The compounds, compositions and methods of these inventions are of particular use for treating neuropathic or inflammatory pain by the inhibition of ion flux through a channel that includes a PN3 (Naᵥ1.8) subunit.

The tetrodotoxin insensitive, peripheral sodium channel Naᵥ1.9, disclosed by Dib-Hajj, S. D., et al. (see Dib-Hajj, S. D., et al., Proc. Natl. Acad. Sci. USA (1998), 95(15):8963-8) was shown to reside solely in the dorsal root ganglia. It has been demonstrated that Naᵥ1.9 underlies neurotrophin (BDNF)-evoked depolarization and excitation, and is the only member of the voltage gated sodium channel superfamily to be shown to be ligand mediated (Blum, R., Kafitz, K. W., Konnerth, A., Nature (2002), 419 (6908):687-93). The limited pattern of expression of this channel has made it a candidate target for the treatment of pain (Lai, J, et al., op. cit.; Wood, J. N., et al., op. cit.; Chung, J. M. et al., op. cit.).

NaX is a putative sodium channel, which has not been shown to be voltage gated. In addition to expression in the lung, heart, dorsal root ganglia, and Schwann cells of the peripheral nervous system, NaX is found in neurons and ependymal cells in restricted areas of the CNS, particularly in the circumventricular organs, which are involved in body-fluid homeostasis (Watanabe, E., et al., J. Neurosci. (2000), 20(20):7743-51). NaX-null mice showed abnormal intakes of hypertonic saline under both water- and salt-depleted conditions. These findings suggest that the NaX plays an important role in the central sensing of body-fluid sodium level and regulation of salt intake behaviour. Its pattern of expression and function suggest it as a target for the treatment of cystic fibrosis and other related salt regulating maladies.

Studies with the sodium channel blocker tetrodotoxin (TTX) used to lower neuron activity in certain regions of the brain, indicate its potential use in the treatment of addiction. Drug-paired stimuli elicit drug craving and relapse in addicts and drug-seeking behavior in rats. The functional integrity of the basolateral amygdala (BLA) is necessary for reinstatement of cocaine-seeking behaviour elicited by cocaine-conditioned stimuli, but not by cocaine itself. BLA plays a similar role in reinstatement of heroin-seeking behavior. TTX-induced inactivation of the BLA on conditioned and heroin-primed reinstatement of extinguished heroin-seeking behaviour in a rat model (Fuchs, R. A. and See, R. E., Psychopharmacology (2002) 160(4):425-33).

This closely related family of proteins has long been recognised as targets for therapeutic intervention. Sodium channels are targeted by a diverse array of pharmacological agents. These include neurotoxins, antiarrhythmics, anticonvulsants and local anesthetics (Clare, J. J., et al., Drug Discovery Today (2000) 5:506-520). All of the current pharmacological agents that act on sodium channels have receptor sites on the alpha subunits. At least six distinct receptor sites for neurotoxins and one receptor site for local anesthetics and related drugs have been identified (Cestèle, S. et al., Biochimie (2000), Vol. 82:883-892).

The small molecule sodium channel blockers or the local anesthetics and related antiepileptic and antiarrhythmic drugs, interact with overlapping receptor sites located in the inner cavity of the pore of the sodium channel (Catterall, W. A., Neuron (2000), 26:13-25). Amino acid residues in the S6 segments from at least three of the four domains contribute to this complex drug receptor site, with the IVS6 segment playing the dominant role. These regions are highly conserved and as such most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrignine, phenytoin and carbamazepine) and certain cardiac arrhythmias (e.g. lignocaine, tocainide and mexiletine). However, the potency and therapeutic index of these blockers is not optimal and have limited the usefulness of these compounds in a variety of therapeutic areas where a sodium channel blocker would be ideally suited.

Management of Acute and Chronic Pain

Drug therapy is the mainstay of management for acute and chronic pain in all age groups, including neonates, infants and children. The pain drugs are classified by the American Pain Society into three main categories; 1) non-opioid analgesics-acetaminophen, and non-steroidal anti-inflammatory drugs (NSAIDs), including salicylates (e.g. aspirin), 2) opioid analgesics and 3) co-analgesics.

Non-opioid analgesics such as acetaminophen and NSAIDs are useful for acute and chronic pain due to a variety of causes including surgery, trauma, arthritis and cancer. NSAIDs are indicated for pain involving inflammation because acetaminophen lacks anti-inflammatory activity. Opioids also lack anti-inflammatory activity. All NSAIDs inhibit the enzyme cyclooxygenase (COX), thereby inhibiting prostaglandin synthesis and reducing the inflammatory pain response. There are at least two COX isoforms, COX-1 and COX-2. Common non-selective COX inhibitors include, ibuprofen and naproxen. Inhibition of COX-1, which is found in platelets, GI tract, kidneys and most other human tissues, is thought to be associated with adverse effects such as gastrointestinal bleeding. The development of selective COX-2 NSAIDs, such as Celecoxib, Valdecoxib and Rofecoxib, have the benefits of non-selective NSAIDs with reduced adverse effect profiles in the gut and kidney. However, evidence now suggests that chronic use of certain selective COX-2 inhibitors can result in an increased risk of stroke occurrence.

The use of opioid analgesics is recommended by the American Pain Society to be initiated based on a pain-directed history and physical that includes repeated pain assessment. Due to the broad adverse effect profiles associated with opiate use, therapy should include a diagnosis, integrated interdisciplinary treatment plan and appropriate ongoing patient monitoring. It is further recommended that opioids be added to non-opioids to manage acute pain and cancer related pain that does not respond to non-opioids alone. Opioid analgesics act as agonists to specific receptors of the mu and kappa types in the central and peripheral nervous system. Depending on the opioid and its formulation or mode of administration it can be of shorter or longer duration. All opioid analgesics have a risk of causing respiratory depression, liver failure, addiction and dependency, and as such are not ideal for long-term or chronic pain management.

A number of other classes of drugs may enhance the effects of opioids or NSAIDSs, have independent analgesic activity in certain situations, or counteract the side effects of analgesics. Regardless of which of these actions the drug has, they are collectively termed “coanalgesics”. Tricyclic antidepressants, antiepileptic drugs, local anaesthetics, glucocorticoids, skeletal muscle relaxants, anti-spasmodil agents, antihistamines, benzodiazepines, caffeine, topical agents (e.g. capsaicin), dextroamphetamine and phenothizines are all used in the clinic as adjuvant therapies or individually in the treatment of pain. The antiepeileptic drugs in particular have enjoyed some success in treating pain conditions. For instance, Gabapentin, which has an unconfirmed therapeutic target, is indicated for neuropathic pain. Other clinical trials are attempting to establish that central neuropathic pain may respond to ion channel blockers such as blockers of calcium, sodium and/or NMDA (N-methyl-D-aspartate) channels. Currently in development are low affinity NMDA channel blocking agents for the treatment of neuropathic pain. The literature provides substantial pre-clinical electrophysiological evidence in support of the use of NMDA antagonists in the treatment of neuropathic pain. Such agents also may find use in the control of pain after tolerance to opioid analgesia occurs, particularly in cancer patients.

Systemic analgesics such as NSAIDs and opioids are to be distinguished from therapeutic agents which are useful only as local analgesics/anaesthetics. Well known local analgesics such as lidocaine and xylocaine are non-selective ion channel blockers which can be fatal when administered systemically. A good description of non-selective sodium channel blockers is found in Madge, D. et al., J. Med. Chem. (2001), 44(2):115-37.

Several sodium channel modulators are known for use as anticonvulsants or antidepressants, such as carbamazepine, amitriptyline, lamotrigine and riluzole, all of which target brain tetradotoxin-sensitive (TTX-S) sodium channels. Such TTX-S agents suffer from dose-limiting side effects, including dizziness, ataxia and somnolence, primarily due to action at TTX-S channels in the brain.

Sodium Channels Role in Pain

Sodium channels play a diverse set of roles in maintaining normal and pathological states, including the long recognized role that voltage gated sodium channels play in the generation of abnormal neuronal activity and neuropathic or pathological pain (Chung, J. M. et al.). Damage to peripheral nerves following trauma or disease can result in changes to sodium channel activity and the development of abnormal afferent activity including ectopic discharges from axotomised afferents and spontaneous activity of sensitized intact nociceptors. These changes can produce long-lasting abnormal hypersensitivity to normally innocuous stimuli, or allodynia. Examples of neuropathic pain include, but are not limited to post-herpetic neuralgia, trigeminal neuralgia, diabetic neuropathy, chronic lower back pain, phantom limb pain, and pain resulting from cancer and chemotherapy, chronic pelvic pain, complex regional pain syndrome and related neuralgias.

There has been some degree of success in treating neuropathic pain symptoms by using medications, such as gabapentin, and more recently pregabalin, as short-term, first-line treatments. However, pharmacotherapy for neuropathic pain has generally had limited success with little response to commonly used pain reducing drugs, such as NSAIDS and opiates. Consequently, there is still a considerable need to explore novel treatment modalities.

There remains a limited number of potent effective sodium channel blockers with a minimum of adverse events in the clinic. There is also an unmet medical need to treat neuropathic pain and other sodium channel associated pathological states effectively and without adverse side effects. The present invention provides compounds, methods of use and compositions that include these compounds to meet these critical needs.

### SUMMARY OF THE INVENTION

The present invention is directed to heterocyclic compounds that are useful for the treatment and/or prevention of sodium channel-mediated diseases or conditions, such as pain. The compounds of the present invention are also useful for the treatment of other sodium channel-mediated diseases or conditions, including, but not limited to central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome.

Accordingly, in one aspect, the invention provides compounds of formula (I): 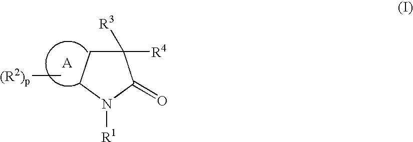
wherein:

p is 0, 1, 2, 3 or 4; 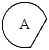
is a fused heteroaryl ring or a fused heterocyclyl ring;

R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;

or R¹ is aralkyl substituted by —C(O)N(R⁷)R⁸ where:

R⁷ is hydrogen, alkyl, aryl or aralkyl; and

R⁸ is hydrogen, alkyl, haloalkyl, —R¹⁰—CN, —R¹⁰—OR⁶, —R¹⁰—N(R⁵)R⁶, aryl, aralkyl, cycloalkyl, cycloalkylalkyl, heterocyclyl, heterocyclylalkyl, heteroaryl, heteroarylalkyl;

or R⁷ and R⁸, together with the nitrogen to which they are attached, form a heterocyclyl or heteroaryl;

and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;

or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;

or R¹ is —R¹⁰—N(R¹¹)R¹², —R¹⁰—N(R¹³)C(O)R¹² or —R¹⁰—N(R¹¹)C(O)N(R¹¹)R¹² where:

each R¹¹ is hydrogen, alkyl, aryl or aralkyl;

each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;

R¹³ is hydrogen, alkyl, aryl, arakyl or —C(O)R⁶;

and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;

or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;

each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵, —C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(═N—CN)N(R⁵)R⁶,

wherein each m is independently 0, 1, or 2 and each n is independently 1 or 2;

and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;

R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶; —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,

wherein X is bromo or chloro, each m is independently 0, 1, or 2 and each n is independently 1 or 2; and

wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a heterocyclyl optionally substituted by alkyl, haloalkyl or —R⁹—OR⁶);

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl;

each R⁹ is a direct bond or an optionally substituted straight or branched alkylene chain, an optionally substituted straight or branched alkenylene chain or an optionally substituted straight or branched alkynylene chain; and

each R¹⁰ is an optionally substituted straight or branched alkylene chain, an optionally substituted straight or branched alkenylene chain or an optionally substituted straight or branched alkynylene chain;

as a stereoisomer, enantiomer, tautomer thereof or mixtures thereof;

or a pharmaceutically acceptable salt, solvate or prodrug thereof.

In another aspect, the invention provides methods for the treatment of pain in a mammal, preferably a human, wherein the methods comprise administering to the mammal in need thereof a therapeutically effective amount of a compound of the invention as set forth above.

In another aspect, the present invention provides a method for treating or lessening the severity of a disease, condition, or disorder where activation or hyperactivity of one or more of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 is implicated in the disease state.

In another aspect, the invention provides methods of treating a range of sodium channel-mediated diseases or conditions, for example, pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke, glaucoma or neural trauma, tachy-arrhythmias, atrial fibrillation and ventricular fibrillation.

In another aspect, the invention provides methods of treating a range of sodium channel-mediated disease or condition through inhibition of ion flux through a voltage-dependent sodium channel in a mammal, preferably a human, wherein the methods comprise administering to the mammal in need thereof a therapeutically effective amount of a compound of the invention as set forth above.

In another aspect, the invention provides pharmaceutical compositions comprising the compounds of the invention, as set forth above, and pharmaceutically acceptable excipients. In one embodiment, the present invention relates to a pharmaceutical composition comprising a compound of the invention in a pharmaceutically acceptable carrier and in an amount effective to treat diseases or conditions related to pain when administered to an animal, preferably a mammal, most preferably a human.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy. In one embodiment, the present invention relates to a pharmaceutical composition combining compounds of the present invention with established or future therapies for the indications listed in the invention.

### DETAILED DESCRIPTION OF THE INVENTION

### DEFINITIONS

Certain chemical groups named herein are preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

Accordingly, as used in the specification and appended claims, unless specified to the contrary, the following terms have the meaning indicated:

“Amino” refers to the —NH₂ radical.

“Cyano” refers to the —CN radical.

“Hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“Alkenyl” refers to a straight or branched hydrocarbon chain radical group consisting solely of carbon and hydrogen atoms, containing at least one double bond, having from two to twelve carbon atoms, preferably one to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁵, —OC(O)—R¹⁵, —N(R¹⁵)₂, —C(O)R¹⁵, —C(O)OR¹⁵, —C(O)N(R¹⁵)₂, —N(R¹⁵)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁵, —OC(O)—R¹⁵, —N(R¹⁵)₂, —C(O)R¹⁵, —C(O)OR¹⁵, —C(O)N(R¹⁵)₂, —N(R¹⁵)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁵, —OC(O)—R¹⁵, —N(R¹⁵)₂, —C(O)R¹⁵, —C(O)OR¹⁵, —C(O)N(R¹⁵)₂, —N(R¹⁵)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, an alkynylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁵, —OC(O)—R¹⁵, —N(R¹⁵)₂, —C(O)R¹⁵, —C(O)OR¹⁵, —C(O)N(R¹⁵)₂, —N(R¹⁵)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“Alkynyl” refers to a straight or branched hydrocarbon chain radical group consisting solely of carbon and hydrogen atoms, containing at least one triple bond, having from two to twelve carbon atoms, preferably one to eight carbon atoms and which is attached to the rest of the molecule by a single bond, e.g., ethynyl, propynyl, butynyl, pentynyl, hexynyl, and the like. Unless stated otherwise specifically in the specification, an alkynyl group may be optionally substituted by one of the following groups: alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —OR¹⁵, —OC(O)—R¹⁵, —N(R¹⁵)₂, —C(O)R¹⁵, —C(O)OR¹⁵, —C(O)N(R¹⁵)₂, —N(R¹⁵)C(O)OR¹⁷, —N(R¹⁵)C(O)R¹⁷, —N(R¹⁵)S(O)ₜR¹⁷ (where t is 1 to 2), —S(O)ₜOR¹⁷ (where t is 1 to 2), —S(O)ₜR¹⁷ (where t is 0 to 2), and —S(O)ₜN(R¹⁵)₂ (where t is 1 to 2) where each R¹⁵ is independently hydrogen, alkyl, haloalkyl, cycloalkyl, cycloalkylalkyl, aryl, aralkyl, heterocyclyl, heterocylylalkyl, heteroaryl or heteroarylalkyl; and each R¹⁷ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“Alkoxy” refers to a radical of the formula —ORₐ where Rₐ is an alkyl radical as defined above containing one to twelve carbon atoms. The alkyl part of the alkoxy radical may be optionally substituted as defined above for an alkyl radical.

“Alkoxyalkyl” refers to a radical of the formula —Rₐ—O—Rₐ where each Rₐ is independently an alkyl radical as defined above. The oxygen atom may be bonded to any carbon in either alkyl radical. Each alkyl part of the alkoxyalkyl radical may be optionally substituted as defined above for an alkyl group.

“Aryl” refers to aromatic monocyclic or multicyclic hydrocarbon ring system consisting only of hydrogen and carbon and containing from 6 to 19 carbon atoms, where the ring system may be partially or fully saturated. Aryl groups include, but are not limited to groups such as fluorenyl, phenyl and naphthyl. Unless stated otherwise specifically in the specification, the term “aryl” or the prefix “ar-” (such as in “aralkyl”) is meant to include aryl radicals optionally substituted by one or more substituents independently selected from the group consisting of alkyl, akenyl, halo, haloalkyl, haloalkenyl, cyano, nitro, aryl, heteroaryl, heteroarylalkyl, —R¹⁶—OR¹⁵, —R¹⁶—OC(O)—R¹⁵, —R¹⁶—N(R¹⁵)₂, —R¹⁶—C(O)R¹⁵, —R¹⁶—C(O)OR¹⁵, —R¹⁶—C(O)N(R¹⁵)₂, —R¹⁶—N(R¹⁵)C(O)OR¹⁷, —R¹⁶—N(R¹⁵)C(O)R¹⁷, —R¹⁶—N(R¹⁵)S(O)ₜR¹⁷ (where t is 1 to 2), —R¹⁶—S(O)ₜOR¹⁷ (where t is 1 to 2), —R¹⁶—S(O)ₜR¹⁷ (where t is 0 to 2), and —R¹⁶—S(O)ₜN(R¹⁵)₂ (where t is 1 to 2) where each R¹⁵ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁶ is independently a direct bond or a straight or branched alkylene or alkenylene chain; and each R¹⁷ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“Aralkyl” refers to a radical of the formula —RₐR_{b where} Rₐ is an alkyl radical as defined above and R_{b} is one or more aryl radicals as defined above, e.g., benzyl, diphenylmethyl and the like. The aryl radical(s) may be optionally substituted as described above.

“Aryloxy” refers to a radical of the formula —OR_{b} where R_{b} is an aryl group as defined above. The aryl part of the aryloxy radical may be optionally substituted as defined above.

“Aralkenyl” refers to a radical of the formula —R_{c}R_{b} where R_{c} is an alkenyl radical as defined above and R_{b} is one or more aryl radicals as defined above, which may be optionally substituted as described above. The aryl part of the aralkenyl radical may be optionally substituted as described above for an aryl group. The alkenyl part of the aralkenyl radical may be optionally substituted as defined above for an alkenyl group.

“Aralkyloxy” refers to a radical of the formula —OR_{b} where R_{b} is an aralkyl group as defined above. The aralkyl part of the aralkyloxy radical may be optionally substituted as defined above.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantine, norbornane, decalinyl, 7,7-dimethyl-bicyclo[2.2.1]heptanyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R¹⁶—OR¹⁵, —R¹⁶—OC(O)—R¹⁵, —R¹⁶—N(R¹⁵)₂, —R¹⁶—C(O)R¹⁵, —R¹⁶—C(O)OR¹⁵, —R¹⁶—C(O)N(R¹⁵)₂, —R¹⁶—N(R¹⁵)C(O)OR¹⁷, —R¹⁶—N(R¹⁵)C(O)R¹⁷, —R¹⁶—N(R¹⁵)S(O)ₜR¹⁷ (where t is 1 to 2), —R¹⁶—S(O)ₜOR¹⁷ (where t is 1 to 2), —R¹⁶—S(O)ₜR¹⁷ (where t is 0 to 2), and —R¹⁶—S(O)ₜN(R¹⁵)₂ (where t is 1 to 2) where each R¹⁵ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁶ is independently a direct bond or a straight or branched alkylene or alkenylene chain; and each R¹⁷ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Heterocyclyl” or “heterocyclyl ring” refers to a stable 3- to 18-membered non-aromatic ring radical which consists of two to seventeen carbon atoms and from one to ten heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁶—OR¹⁵, —R¹⁶—OC(O)—R¹⁵, —R¹⁶—N(R¹⁵)₂, —R¹⁶—C(O)R¹⁵, —R¹⁶—C(O)OR¹⁵, —R¹⁶—C(O)N(R¹⁵)₂, —R¹⁶—N(R¹⁵)C(O)OR¹⁷, —R¹⁶—N(R¹⁵)C(O)R¹⁷, —R¹⁶—N(R¹⁵)S(O)ₜR¹⁷ (where t is 1 to 2), —R¹⁶—S(O)ₜOR¹⁷ (where t is 1 to 2), —R¹⁶—S(O)ₜR¹⁷ (where t is 0 to 2), and —R¹⁶—S(O)ₜN(R¹⁵)₂ (where t is 1 to 2) where each R¹⁵ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁶ is independently a direct bond or a straight or branched alkylene or alkenylene chain; and each R¹⁷ is alkyl, alkenyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“N-heterocyclyl” is a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The alkyl part of the heterocyclylalkyl radical may be optionally substituted as defined above for an alkyl group. The heterocyclyl part of the heterocyclylalkyl radical may be optionally substituted as defined above for a heterocyclyl group.

“Heteroaryl” or “heteroaryl ring” refers to a 5- to 18-membered aromatic ring radical which consists of three to seventeen carbon atoms and from one to ten heteroatoms selected from the group consisting of nitrogen, oxygen and sulfur. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzo-1,3-dioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, alkenyl, halo, haloalkyl, haloalkenyl, cyano, oxo, thioxo, nitro, oxo, aryl, aralkyl, cycloalkyl, cycloalkylalkyl, heterocyclyl, heterocyclylalkyl, heteroaryl, heteroarylalkyl, —R¹⁶—OR¹⁵, —R¹⁶—OC(O)—R¹⁵, —R¹⁶—N(R¹⁵)₂, —R¹⁶—C(O)R¹⁵, —R¹⁶—C(O)OR¹⁵, —R¹⁶—C(O)N(R¹⁵)₂, —R¹⁶—N(R¹⁵)C(O)OR¹⁷, —R¹⁶—N(R¹⁵)C(O)R¹⁷, —R¹⁶—N(R¹⁵)S(O)ₜR¹⁷ (where t is 1 to 2), —R¹⁶—S(O)ₜOR¹⁷ (where t is 1 to 2), —R¹⁶—S(O)ₜR¹⁷ (where t is 0 to 2), and —R¹⁶—S(O)ₜN(R¹⁵)₂ (where t is 1 to 2) where each R¹⁵ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁶ is independently a direct bond or a straight or branched alkylene or alkenylene chain; and each R¹⁷ is alkyl, alkenyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“N-heteroaryl” is a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —RₐR_{f} where Rₐ is an alkyl radical as defined above and R_{f} is a heteroaryl radical as defined above. The heteroaryl part of the heteroarylalkyl radical may be optionally substituted as defined above for a heteroaryl group. The alkyl part of the heteroarylalkyl radical may be optionally substituted as defined above for an alkyl group.

“Heteroarylalkenyl” refers to a radical of the formula —R_{b}R_{f} where R_{b} is an alkenyl radical as defined above and R_{f} is a heteroaryl radical as defined above. The heteroaryl part of the heteroarylalkenyl radical may be optionally substituted as defined above for a heteroaryl group. The alkenyl part of the heteroarylalkenyl radical may be optionally substituted as defined above for an alkenyl group.

“Trihaloalkyl” refers to an alkyl radical, as defined above, that is substituted by three halo radicals, as defined above, e.g., trifluoromethyl. The alkyl part of the trihaloalkyl radical may be optionally substituted as defined above for an alkyl group.

“Trihaloalkoxy” refers to a radical of the formula —OR_{g} where R_{g} is a trihaloalkyl group as defined above. The trihaloalkyl part of the trihaloalkoxy group may be optionally substituted as defined above for a trihaloalkyl group.

“Analgesia” refers to an absence of pain in response to a stimulus that would normally be painful.

“Allodynia” refers to a condition in which a normally innocuous sensation, such as pressure or light touch, is perceived as being extremely painful.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)).

A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of formula (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action on the sodium channels, or binding affinity to pharmacologically important site of action on the sodium channels. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples and Preparations as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically are identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, and rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandetic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of a sodium channel-mediated disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are intermediate compounds of formula (I) and all polymorphs of the aforementioned species and crystal habits thereof.

The chemical naming protocol and structure diagrams used herein are a modified form of the I.U.P.A.C. nomenclature system, using the ACD/Name Version 9.07 software program, wherein the compounds of the invention are named herein as derivatives of the central core structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

Thus, for example, a compound of formula (I) wherein p is 0, R¹ is pentyl, R³ is hydroxy, R⁴ is benzo-1,3-dioxolyl; and 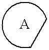
is a fused thienyl ring; 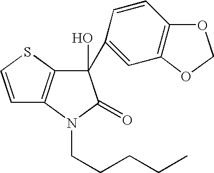
is named herein as 4-(1,3-benzodioxol-5-yl)-4-hydroxy-6-pentyl-4,6-dihydro-5H-thieno[2,3-b]pyrrol-5-one.

### EMBODIMENTS OF THE INVENTION

Of the various aspects of the invention set forth above in the Summary of the Invention, certain embodiments are preferred.

One embodiment is a compound of formula (I), as set forth above in the Summary of the Invention, wherein:

p is 0, 1, 2, 3 or 4; 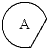
is a fused heteroaryl ring selected from the group consisting of pyrrolyl, pyrazolyl, pyridinyl, pyrimidinyl, thienyl and pyrazinyl;

R¹ is —R⁹—C(O)R , —R⁹—C(O)OR⁶, —R⁹—OR⁶, —R⁹—CN, —R¹⁰—P(O)(OR⁶)₂, —R¹⁰—O—R¹⁰—OR⁶, hydrogen, alkyl, haloalkyl, cycloalkylalkyl, heterocyclylalkyl, aryl (optionally substituted by one or more substituents selected from the group consisting of halo and —R⁹—C(O)OR⁶), aralkyl (optionally substituted by one or more substituents selected from the group consisting of halo, haloalkyl, heteroaryl, —R⁹—OR⁶and —R⁹—C(O)OR⁶), heteroaryl (optionally substituted by one or more substituents selected from the group consisting of alkyl, halo, haloalkyl and —R⁹—OR⁶), or heteroarylalkyl (optionally substituted by one or more substituents selected from the group consisting of alkyl, halo, haloalkyl and —R⁹—OR⁶);

each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵; —R⁹—C(O)OR⁶, —R⁹—C(O)N(R⁵)R⁶, —N(R⁶)C(O)R⁵,

wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;

or two adjacent R² groups, together with the fused heteroaryl ring atoms to which they are directly attached, may form a fused ring selected from cycloalkyl, aryl, heterocyclyl and heteroaryl, and the remaining R² groups, if present, are as described above;

R³ is independently selected from the group consisting of hydrogen, halo, haloalkyl, —R⁹—OR⁶, —R⁹—OC(O)R⁶, —R⁹—CN, —R⁹—N(R⁵)R⁶, —R⁹—C(O)R⁵, —R⁹—C(O)X, —R⁹—C(O)OR⁶ and —N(R⁶)C(O)OR⁶, wherein X is chloro or bromo;

R⁴ is independently selected from the group consisting of alkyl, aryl, aralkyl, aralkynyl, heteroaryl, heteroarylalkyl, —R⁹—C(O)R⁵, —N(R⁶)C(O)N(R⁵)R⁶, —R⁹—NO₂, —R⁹—N(R⁵)R⁶, —R⁹—C(O)OR⁶, —R⁹—N(R⁶)C(O)OR⁶ and —Si(R⁶)₃,

wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶);

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl;

each R⁹ is a direct bond or an optionally substituted straight or branched alkylene chain, an optionally substituted straight or branched alkenylene chain or an optionally substituted straight or branched alkynylene chain; and

each R¹⁰ is an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 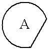
is a fused heteroaryl ring selected from the group consisting of pyrrolyl, pyrazolyl, pyridinyl, pyrimidinyl, thienyl and pyrazinyl;

R¹ is alkyl, aryl or aralkyl, where each of the aryl or aralkyl group for R¹ is optionally substituted by one or more substituents selected from the group consisting of halo, haloalkyl, heteroaryl, —R⁹—OR⁶ and —R⁹—C(O)OR⁶;

each R² is independently selected from the group consisting of alkyl, halo, aryl, heteroaryl and —R⁹—OR⁶,

wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

R³ is hydrogen, halo, —R⁹—OR⁶ or —R⁹—OC(O)R⁶;

R⁴ is independently selected from the group consisting of alkyl, aryl, aralkynyl, heteroaryl, heteroarylalkyl, —R⁹—C(O)R⁵, —N(R⁶)C(O)N(R⁵)R⁶, —R⁹—NO₂, —R⁹—N(R⁵)R⁶, —R⁹—C(O)OR⁶ and —Si(R⁶)₃,

wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 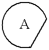
is a fused heteroaryl ring selected from the group consisting of pyrrolyl, pyrazolyl, pyridinyl, pyrimidinyl, thienyl and pyrazinyl;

R¹ is alkyl, aryl or aralkyl, where each of the aryl or aralkyl group for R¹ is optionally substituted by one or more substituents selected from the group consisting of halo, haloalkyl, heteroaryl, —R⁹—OR⁶ and —R⁹—C(O)OR⁶;

each R² is independently selected from the group consisting of alkyl, halo, aryl, heteroaryl and —R⁹—OR⁶,

wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

R³ is hydrogen, halo, —R⁹—OR⁶ or —R⁹—OC(O)R⁶;

R⁴ is —R⁹—C(O)R⁵;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 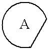
is a fused heteroaryl ring selected from the group consisting of pyrrolyl, pyrazolyl, pyridinyl, pyrimidinyl, thienyl and pyrazinyl;

R¹ is aralkyl (optionally substituted by one or more substituents selected from the group consisting of halo, haloalkyl, heteroaryl, —R⁹—OR⁶ and —R⁹—C(O)OR⁶);

each R² is independently selected from the group consisting of alkyl, halo, aryl, heteroaryl and —R⁹—OR⁶,

wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;

R³ is hydrogen, halo, —R⁹—OR⁶ or —R⁹—OC(O)R⁶;

R⁴ is heterocyclylalkyl, heteroaryl or heteroarylalkyl, each optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 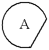
is a fused heteroaryl ring selected from the group consisting of pyrrolyl, pyrazolyl, pyridinyl, pyrimidinyl, thienyl and pyrazinyl;

R¹ is aralkyl (optionally substituted by one or more substituents selected from the group consisting of halo, haloalkyl, heteroaryl, —R⁹—OR⁶ and —R⁹—C(O)OR⁶);

each R² is each independently selected from the group consisting of alkyl, halo, phenyl, benzodioxolyl and —R⁹—OR⁶,

R³ is hydrogen, halo, —R⁹—OR⁶ or —R⁹—OC(O)R⁶;

R⁴ is heterocyclylalkyl, heteroaryl or heteroarylalkyl, each optionally substituted by one or more substituents selected from the group consisting of halo, heterocyclyl, and —R⁹—OR⁶;

each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0; 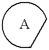
is a fused heteroaryl ring selected from the group consisting of pyrrolyl, pyrazolyl, pyridinyl, pyrimidinyl, thienyl and pyrazinyl;

R¹ is aralkyl (optionally substituted by one or more substituents selected from the group consisting of halo, haloalkyl, heteroaryl, —R⁹—OR⁶ and —R⁹—C(O)OR⁶);

R³ is —R⁹—OR⁶;

R⁴ is aryl, aralkyl or aralkynyl,

wherein each of the aryl, aralkyl and aralkynyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR , —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0; 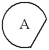
is a fused heteroaryl ring selected from the group consisting of pyrrolyl, pyrazolyl, pyridinyl, pyrimidinyl, thienyl and pyrazinyl;

R¹ is aralkyl (optionally substituted by one or more substituents selected from the group consisting of halo, haloalkyl, heteroaryl, —R⁹—OR⁶ and —R⁹—C(O)OR⁶);

R³ is —R⁹—OR⁶;

R⁴ is aryl, aralkyl or aralkynyl,

wherein each of the aryl, aralkyl and aralkynyl groups for R⁴ is optionally substituted by one or more substituents selected from the group consisting of halo, oxo and —R⁹—OR⁶;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain.

Another embodiment of the invention is a compound of formula (I), as set forth above in the Summary of the Invention, wherein:

p is 0, 1, 2, 3 or 4; 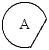
is a fused heteroaryl ring selected from the group consisting of pyrrolyl, pyrazolyl, pyridinyl, pyrimidinyl, thienyl and pyrazinyl;

R¹ is hydrogen, alkyl, haloalkyl or cycloalkylalkyl;

each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵; —R⁹—C(O)OR⁶, —R⁹—C(O)N(R⁵)R⁶, —N(R⁶)C(O)R⁵,

wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

or two adjacent R² groups, together with the heteroaryl ring atoms to which they are directly attached, may form a fused ring selected from cycloalkyl, aryl, heterocyclyl and heteroaryl, and the remaining R² groups, if present, are as described above;

R³is hydrogen, halo or —R⁹—OR⁶;

R⁴is independently selected from the group consisting of alkyl, aryl, aralkynyl, heteroaryl, heteroarylalkyl, —R⁹—C(O)R⁵, —R⁹—N(R⁶)C(O)OR⁶, —N(R⁶)C(O)N(R⁵)R⁶, —R⁹—NO₂, —R⁹—N(R⁵)R⁶, —R⁹—C(O)OR⁶, and —Si(R⁶)₃,

wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 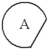
is a fused heteroaryl ring selected from the group consisting of pyrrolyl, pyrazolyl, pyridinyl, pyrimidinyl, thienyl and pyrazinyl

R¹ is hydrogen, alkyl, haloalkyl or cycloalkylalkyl;

each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;

—R⁹—C(O)OR⁶, —R⁹—C(O)N(R⁵)R⁶, —N(R⁶)C(O)R⁵,

wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

or two adjacent R² groups, together with the heteroaryl ring atoms to which they are directly attached, may form a fused ring selected from cycloalkyl, aryl, heterocyclyl and heteroaryl, and the remaining R² groups, if present, are as described above;

R³ is hydrogen or —R⁹—OR⁶;

R⁴ is heteroaryl optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 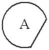
is a fused heteroaryl ring selected from the group consisting of pyrrolyl, pyrazolyl, pyridinyl, pyrimidinyl, thienyl and pyrazinyl;

R¹ is alkyl;

each R² is independently selected from the group consisting of alkyl, halo, haloalkyl and —R⁹—OR⁶;

R³ is hydrogen or —R⁹—OR⁶;

R⁴ is heteroaryl optionally substituted by one or more substituents selected from the group consisting of halo, —R⁹—OR⁶ and —N(R⁶)C(O)R⁵;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 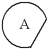
is a fused heteroaryl ring selected from the group consisting of pyrrolyl, pyrazolyl, pyridinyl, pyrimidinyl, thienyl and pyrazinyl;

R¹ is alkyl;

each R² is independently selected from the group consisting of alkyl, halo, haloalkyl and —R⁹OR⁶;

R³ is hydrogen or —R⁹—OR⁶;

R⁴ is benzodioxolyl optionally substituted by one or more substituents selected from the group consisting of halo and —R⁹—OR⁶;

each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 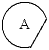
is a fused heteroaryl ring selected from the group consisting of pyrrolyl, pyrazolyl, pyridinyl, pyrimidinyl, thienyl and pyrazinyl;

R¹ is alkyl;

each R² is independently selected from the group consisting of alkyl, halo, haloalkyl and —R⁹—OR⁶;

R³ is hydrogen, halo or —R⁹—OR⁶;

R⁴ is independently selected from the group consisting of —R⁹—C(O)R⁵ and —R⁹—N(R⁶)C(O)OR⁶;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0; 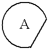
is a fused heteroaryl ring selected from the group consisting of pyrrolyl, pyrazolyl, pyridinyl, pyrimidinyl, thienyl and pyrazinyl;

R¹ is alkyl or aralkyl (optionally substituted by one or more substituents selected from the group consisting of halo, haloalkyl, —R⁹—OR⁶, heteroaryl and —R⁹—C(O)OR⁶);

R³ is —R⁹—C(O)X, —R⁹—C(O)OR⁶ and —R⁹—C(O)N(R⁵)R⁶ where X is bromo or chloro;

R⁴ is independently selected from the group consisting of —R⁹—C(O)R⁵ and heteroaryl optionally substituted by one or more substituents selected from the group consisting of halo and R⁹—OR⁶;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 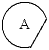
is a fused heteroaryl ring selected from the group consisting of pyrrolyl, pyrazolyl, pyridinyl, pyrimidinyl, thienyl and pyrazinyl;

R¹ is alkyl or aralkyl optionally substituted by one or more substituents selected from the group consisting of halo and —R⁹—C(O)OR⁶;

each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;

—R⁹—C(O)OR⁶, —R⁹—C(O)N(R⁵)R⁶, —N(R⁶)C(O)R⁵,

wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;

or two adjacent R² groups, together with the heteroaryl ring atoms to which they are directly attached, may form a fused ring selected from cycloalkyl, aryl, heterocyclyl and heteroaryl, and the remaining R² groups, if present, are as described above;

R³ and R⁴ together form ═NS(O)₂R⁶, ═N—R¹⁵, ═N—O—R⁶ or ═R^{9a}—C(O)R⁶,

where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 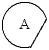
is a fused heteroaryl ring selected from the group consisting of pyrrolyl, pyrazolyl, pyridinyl, pyrimidinyl, thienyl and pyrazinyl;

R¹ is alkyl or aralkyl optionally substituted by one or more substituents selected from the group consisting of halo and —R⁹—C(O)OR⁶;

each R² is independently selected from the group consisting of alkyl, halo and haloalkyl;

or two adjacent R² groups, together with the heteroaryl ring atoms to which they are directly attached, may form a fused ring selected from cycloalkyl, aryl, heterocyclyl and heteroaryl, and the remaining R² groups, if present, are as described above;

R³ and R⁴ together form ═NS(O)₂R⁶, ═N—R¹⁵, ═N—O—R⁶ or ═R⁹a—C(O)R⁶,

where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶;

each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and

each R⁹ is a direct bond or an optionally substituted straight or branched alkylene chain, an optionally substituted straight or branched alkenylene chain or an optionally substituted straight or branched alkynylene chain.

Another embodiment of the invention is a compound of formula (I), as set forth above in the Summary of the Invention, wherein:

p is 0, 1, 2, 3 or 4; 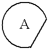
is a fused heteroaryl ring selected from the group consisting of pyridinyl, pyrimidinyl, thienyl and pyrazinyl;

R¹ is alkyl;

each R² is independently selected from the group consisting of alkyl, halo, haloalkyl and —R⁹—OR⁶;

R³ is independently selected from the group consisting of halo, —R⁹—CN, —R⁹—N(R⁵)R⁶ and —N(R⁶)C(O)OR⁶;

R⁴ is heteroaryl optionally substituted by one or more substituents selected from the group consisting of alkyl, halo, haloalkyl, and —R⁹—OR⁶;

each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;

or when R⁵ and R⁶ are each attached to the same nitrogen atom, then R⁵ and R⁶, together with the nitrogen atom to which they are attached, may form a N-heterocyclyl or N-heteroaryl; and

each R⁹ is a direct bond or an optionally substituted straight or branched alkylene chain, an optionally substituted straight or branched alkenylene chain or an optionally substituted straight or branched alkynylene chain.

Specific embodiments of the compounds of formula (I) are described in more detail below in the Preparation of the Compounds of the Invention.

### UTILITY AND TESTING OF THE COMPOUNDS OF THE INVENTION

The present invention relates to compounds, pharmaceutical compositions and methods of using the compounds and pharmaceutical compositions for the treatment of sodium channel-mediated diseases, preferably diseases related to pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome, by administering to a patient in need of such treatment an effective amount of a sodium channel blocker modulating, especially inhibiting, agent.

In general, the present invention provides a method for treating a patient for, or protecting a patient from developing, a sodium channel-mediated disease, especially pain, comprising administering to an animal, such as a mammal, especially a human patient in need thereof, a therapeutically effective amount of a compound of the invention or a pharmaceutical composition comprising a compound of the invention wherein the compound modulates the activity of one or more voltage-dependent sodium channels.

The general value of the compounds of the invention in mediating, especially inhibiting, the sodium channel ion flux can be determined using the assays described below in the Biological Assays section. Alternatively, the general value of the compounds in treating conditions and diseases may be established in industry standard animal models for demonstrating the efficacy of compounds in treating pain. Animal models of human neuropathic pain conditions have been developed that result in reproducible sensory deficits (allodynia, hyperalgesia, and spontaneous pain) over a sustained period of time that can be evaluated by sensory testing. By establishing the degree of mechanical, chemical, and temperature induced allodynia and hyperalgesia present, several physiopathological conditions observed in humans can be modeled allowing the evaluation of pharmacotherapies.

In rat models of peripheral nerve injury, ectopic activity in the injured nerve corresponds to the behavioural signs of pain. In these models, intravenous application of the sodium channel blocker and local anesthetic lidocaine can suppress the ectopic activity and reverse the tactile allodynia at concentrations that do not affect general behaviour and motor function (Mao, J. and Chen, L. L, Pain (2000), 87:7-17). Allimetric scaling of the doses effective in these rat models, translates into doses similar to those shown to be efficacious in humans (Tanelian, D. L. and Brose, W. G., Anesthesiology (1991), 74(5):949-951.). Furthermore, Lidoderm®, lidocaine applied in the form of a dermal patch, is currently an FDA approved treatment for post-herpetic neuralgia (Devers, A. and Glaler, B. S., Clin. J. Pain (2000), 16(3):205-8).

Sodium channel blockers have clinical uses in addition to pain. Epilepsy and cardiac arrhythmias are often targets of sodium channel blockers. Recent evidence from animal models suggest that sodium channel blockers may also be useful for neuroprotection under ischaemic conditions caused by stroke or neural trauma and in patients with multiple sclerosis (MS) (Clare, J. J. et al., op. cit. and Anger, T: et al., op. cit.).

The compounds of the invention modulate, preferably inhibit, ion flux through a voltage-dependent sodium channel in a mammal, especially in a human. Any such modulation, whether it be partial or complete inhibition or prevention of ion flux, is sometimes referred to herein as “blocking” and corresponding compounds as “blockers”. In general, the compounds of the invention modulates the activity of a sodium channel downwards, inhibits the voltage-dependent activity of the sodium channel, and/or reduces or prevents sodium ion flux across a cell membrane by preventing sodium channel activity such as ion flux.

The compounds of the instant invention are sodium channel blockers and are therefore useful for treating diseases and conditions in humans and other organisms, including all those human diseases and conditions which are the result of aberrant voltage-dependent sodium channel biological activity or which may be ameliorated by modulation of voltage-dependent sodium channel biological activity.

As defined herein, a sodium channel-mediated disease or condition refers to a disease or condition which is ameliorated upon modulation of the sodium channel and includes, but is not limited to, pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome.

A sodium channel-mediated disease or condition also includes pain associated with HIV, HIV treatment induced neuropathy, trigeminal neuralgia, glossopharyngeal neuralgia, neuropathy secondary to metastatic infiltration, adiposis dolorosa, thalamic lesions, hypertension, autoimmune disease, asthma, drug addiction (e.g. opiate, benzodiazepine, amphetamine, cocaine, alcohol, butane inhalation), Alzheimer, dementia, age-related memory impairment, Korsakoff syndrome, restenosis, urinary dysfunction, incontinence, parkinson's disease, cerebrovascular ischemia, neurosis, gastrointestinal disease, sickle cell anemia, transplant rejection, heart failure, myocardial infarction, reperfusion injury, intermittant claudication, angina, convulsion, respiratory disorders, cerebral or myocardial ischemias, long-QT syndrome, Catecholeminergic polymorphic ventricular tachycardia, ophthalmic diseases, spasticity, spastic paraplegia, myopathies, myasthenia gravis, paramyotonia congentia, hyperkalemic periodic paralysis, hypokalemic periodic paralysis, alopecia, anxiety disorders, psychotic disorders, mania, paranoia, seasonal affective disorder, panic disorder, obsessive compulsive disorder (OCD), phobias, autism, Aspergers Syndrome, Retts syndrome, disintegrative disorder, attention deficit disorder, aggressivity, impulse control disorders, thrombosis, pre clampsia, congestive cardiac failure, cardiac arrest, Freidrich's ataxia, Spinocerebellear ataxia, myelopathy, radiculopathy, systemic lupus erythamatosis, granulomatous disease, olivo-ponto-cerebellar atrophy, spinocerebellar ataxia, episodic ataxia, myokymia, progressive pallidal atrophy, progressive supranuclear palsy and spasticity, traumatic brain injury, cerebral oedema, hydrocephalus injury, spinal cord injury, anorexia nervosa, bulimia, Prader-Willi syndrome, obesity, optic neuritis, cataract, retinal haemorrhage, ischaemic retinopathy, retinitis pigmentosa, acute and chronic glaucoma, macular degeneration, retinal artery occlusion, Chorea, Huntington's chorea, cerebral edema, proctitis, post-herpetic neuralgia, eudynia, heat sensitivity, tosarcoidosis, irritable bowel syndrome, Tourette syndrome, Lesch-Nyhan Syndrome, Brugado syndrome, Liddle syndrome, Crohns disease, multiple sclerosis and the pain associated with multiple sclerosis (MS), amyotrophic lateral sclerosis (ALS), disseminated sclerosis, diabetic neuropathy, peripheral neuropathy, charcot marie tooth syndrome, arthritic, rheumatoid arthritis, osteoarthritis, chondrocalcinosis, atherosclerosis, paroxysmal dystonia, myasthenia syndromes, myotonia, myotonic dystrophy, muscular dystrophy, malignant hyperthermia, cystic fibrosis, pseudoaldosteronism, rhabdomyolysis, mental handicap, hypothyroidism, bipolar depression, anxiety, schizophrenia, sodium channel toxin related illnesses, familial erythermalgia, primary erythermalgia, rectal pain, cancer, narcotic drug addiction, epilepsy, partial and general tonic seizures, febrile seizures, absence seizures (petit mal), myoclonic seizures, atonic seizures, clonic seizures, Lennox Gastaut, West Syndome (infantile spasms), multiresistant seizures, seizure prophylaxis (anti-epileptogenic), familial Mediterranean fever syndrome, gout, restless leg syndrome, arrhythmias, fibromyalgia, neuroprotection under ischaemic conditions caused by stroke or neural trauma, tachy-arrhythmias, atrial fibrillation and ventricular fibrillation and as a general or local anaesthetic.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g. musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, pain associated with narcotic drug addiction withdrawal and combinations thereof.

The compounds identified in the instant specification inhibit the ion flux through a voltage-dependent sodium channel. Preferably, the compounds are state or frequency dependent modifers of the sodium channels, having a low affinity for the rested/closed state and a high affinity for the inactivated state. These compounds are h likely to interact with overlapping sites located in the inner cavity of the sodium conducting pore of the channel similar to that described for other state-dependent sodium channel blockers (Cestèle, S., et al., op. cit.). These compounds may also be likely to interact with sites outside of the inner cavity and have allosteric effects on sodium ion conduction through the channel pore.

Any of these consequences may ultimately be responsible for the overall therapeutic benefit provided by these compounds.

The present invention readily affords many different means for identification of sodium channel modulating agents that are useful as therapeutic agents. Identification of modulators of sodium channel can be assessed using a variety of in vitro and in vivo assays, e.g. measuring current, measuring membrane potential, measuring ion flux, (e.g. sodium or guanidinium), measuring sodium concentration, measuring second messengers and transcription levels, and using e.g., voltage-sensitive dyes, radioactive tracers, and patch-clamp electrophysiology.

One such protocol involves the screening of chemical agents for ability to modulate the activity of a sodium channel thereby identifying it as a modulating agent.

A typical assay described in Bean et al., J. General Physiology (1983), 83:613-642, and Leuwer, M., et al., Br. J. Pharmacol. (2004), 141(1):47-54, uses patch-clamp techniques to study the behaviour of channels. Such techniques are known to those skilled in the art, and may be developed, using current technologies, into low or medium throughput assays for evaluating compounds for their ability to modulate sodium channel behaviour.

A competitive binding assay with known sodium channel toxins such as tetrodotoxin, alpha-scorpion toxins, aconitine, BTX and the like, may be suitable for identifying potential therapeutic agents with high selectivity for a particular sodium channel. The use of BTX in such a binding assay is well known and is described in McNeal, E. T., et al., J. Med. Chem. (1985), 28(3):381-8; and Creveling, C. R., et al., Methods in Neuroscience, Vol. 8: Neurotoxins (Conn P M Ed) (1992):25-37, Academic Press, New York.

These assays can be carried out in cells, or cell or tissue extracts expressing the channel of interest in a natural endogenous setting or in a recombinant setting. The assays that can be used include plate assays which measure Na+ influx through surrogate markers such as ¹⁴C-guanidine influx or determine cell depolarization using fluorescent dyes such as the FRET based and other fluorescent assays or a radiolabelled binding assay employing radiolabelled aconitine, BTX, TTX or STX. More direct measurements can be made with manual or automated electrophysiology systems. The guanidine influx assay is explained in more detail below in the Biological Assays section.

Throughput of test compounds is an important consideration in the choice of screening assay to be used. In some strategies, where hundreds of thousands of compounds are to be tested, it is not desirable to use low throughput means. In other cases, however, low throughput is satisfactory to identify important differences between a limited number of compounds. Often it will be necessary to combine assay types to identify specific sodium channel modulating compounds.

Electrophysiological assays using patch clamp techniques is accepted as a gold standard for detailed characterization of sodium channel compound interactions, and as described in Bean et al., op. cit. and Leuwer, M., et al., op. cit. There is a manual low-throughput screening (LTS) method which can compare 2-10 compounds per day; a recently developed system for automated medium-throughput screening (MTS) at 20-50 patches (i.e. compounds) per day; and a technology from Molecular Devices Corporation (Sunnyvale, Calif.) which permits automated high-throughput screening (HTS) at 1000-3000 patches (i.e. compounds) per day.

One automated patch-clamp system utilizes planar electrode technology to accelerate the rate of drug discovery. Planar electrodes are capable of achieving high-resistance, cells-attached seals followed by stable, low-noise whole-cell recordings that are comparable to conventional recordings. A suitable instrument is the PatchXpress 7000A (Axon Instruments Inc, Union City, Calif.). A variety of cell lines and culture techniques, which include adherent cells as well as cells growing spontaneously in suspension are ranked for seal success rate and stability. Immortalized cells (e.g. HEK and CHO) stably expressing high levels of the relevant sodium ion channel can be adapted into high-density suspension cultures.

Other assays can be selected which allow the investigator to identify compounds which block specific states of the channel, such as the open state, closed state or the resting state, or which block transition from open to closed, closed to resting or resting to open. Those skilled in the art are generally familiar with such assays.

Binding assays are also available, however these are of only limited functional value and information content. Designs include traditional radioactive filter based binding assays or the confocal based fluorescent system available from Evotec OAI group of companies (Hamburg, Germany), both of which are HTS.

Radioactive flux assays can also be used. In this assay, channels are stimulated to open with veratridine or aconitine and held in a stabilized open state with a toxin, and channel blockers are identified by their ability to prevent ion influx. The assay can use radioactive ²²[Na] and ¹⁴[C] guanidinium ions as tracers. FlashPlate & Cytostar-T plates in living cells avoids separation steps and are suitable for HTS. Scintillation plate technology has also advanced this method to HTS suitability. Because of the functional aspects of the assay, the information content is reasonably good.

Yet another format measures the redistribution of membrane potential using the FLIPR system membrane potential kit (HTS) available from Molecular Dynamics (a division of Amersham Biosciences, Piscataway, N.J.). This method is limited to slow membrane potential changes. Some problems may result from the fluorescent background of compounds. Test compounds may also directly influence the fluidity of the cell membrane and lead to an increase in intracellular dye concentrations. Still, because of the functional aspects of the assay, the information content is reasonably good.

Sodium dyes can be used to measure the rate or amount of sodium ion influx through a channel. This type of assay provides a very high information content regarding potential channel blockers. The assay is functional and would measure Na+ influx directly. CoroNa Red, SBFI and/or sodium green (Molecular Probes, Inc. Eugene Oreg.) can be used to measure Na influx; all are Na responsive dyes. They can be used in combination with the FLIPR instrument. The use of these dyes in a screen has not been previously described in the literature. Calcium dyes may also have potential in this format.

In another assay, FRET based voltage sensors are used to measure the ability of a test compound to directly block Na influx. Commercially available HTS systems include the VIPR™ II FRET system (Aurora Biosciences Corporation, San Diego, Calif., a division of Vertex-Pharmaceuticals, Inc.) which may be used in conjunction with FRET dyes, also available from Aurora Biosciences. This assay measures sub-second responses to voltage changes. There is no requirement for a modifier of channel function. The assay measures depolarization and hyperpolarizations, and provides ratiometric outputs for quantification. A somewhat less expensive MTS version of this assay employs the FLEXstation™ (Molecular Devices Corporation) in conjunction with FRET dyes from Aurora Biosciences. Other methods of testing the compounds disclosed herein are also readily known and available to those skilled in the art.

These results provide the basis for analysis of the structure-activity relationship (SAR) between test compounds and the sodium channel. Certain substituents on the core structure of the test compound tend to provide more potent inhibitory compounds. SAR analysis is one of the tools those skilled in the art may now employ to identify preferred embodiments of the compounds of the invention for use as therapeutic agents.

Modulating agents so identified are then tested in a variety of in vivo models so as to determine if they alleviate pain, especially chronic pain or other conditions such as arrhythmias and epilepsy with minimal adverse events. The assays described below in the Biological Assays Section are useful in assessing the biological activity of the instant compounds.

Typically, a successful therapeutic agent of the present invention will meet some or all of the following criteria. Oral availability should be at or above 20%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 10 μM, preferably below 1 μM and most preferably below 50 nM. The IC₅₀ (“Inhibitory Concentration—50%”) is a measure of the amount of compound required to achieve 50% inhibition of ion flux through a sodium channel, over a specific time period, in an assay of the invention. Compounds of the present invention in the guanidine influx assay have demonstrated IC-50s ranging from less than a nanomolar to less than 10 micromolar.

In an alternative use of the invention, the compounds of the invention can be used in in vitro or in vivo studies as exemplary agents for comparative purposes to find other compounds also useful in treatment of, or protection from, the various diseases disclosed herein.

Another aspect of the invention relates to inhibiting Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample or a patient, which method comprises administering to the patient, or contacting said biological sample with a compound of formula I or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample is useful for a variety of purposes that are known to one of skill in the art. Examples of such purposes include, but are not limited to, the study of sodium ion channels in biological and pathological phenomena; and the comparative evaluation of new sodium ion channel inhibitors.

### PHARMACEUTICAL COMPOSITIONS OF THE INVENTION AND ADMINISTRATION

The present invention also relates to pharmaceutical composition containing the compounds of the invention disclosed herein. In one embodiment, the present invention relates to a composition comprising compounds of the invention in a pharmaceutically acceptable carrier and in an amount effective to modulate, preferably inhibit, ion flux through a voltage-dependent sodium channel to treat sodium channel mediated diseases, such as pain, when administered to an animal, preferably a mammal, most preferably a human patient.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

Those skilled in the art know how to determine suitable doses of the compounds for use in treating the diseases and conditions contemplated herein. Therapeutic doses are generally identified through a dose ranging study in humans based on preliminary evidence derived from animal studies. Doses must be sufficient to result in a desired therapeutic benefit without causing unwanted side effects for the patient.

A typical regimen for treatment of sodium channel-mediated disease comprises administration of an effective amount over a period of one or several days, up to and including between one week and about six months, or it may be chronic. It is understood that the dosage of a diagnostic/pharmaceutical compound or composition of the invention administered in vivo or in vitro will be dependent upon the age, sex, health, and weight of the recipient, severity of the symptons, kind of concurrent treatment, if any, frequency of treatment, the response of the individual, and the nature of the diagnostic/pharmaceutical effect desired. The ranges of effective doses provided herein are not intended to be limiting and represent preferred dose ranges. However, the most preferred dosage will be tailored to the individual subject, as is understood and determinable by one skilled in the relevant arts. (see, e.g., Berkowet al., eds., The Merck Manual, 16^{th} edition, Merck and Co., Rahway, N. J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18th edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. Effective amounts of a diagnostic pharmaceutical compound or composition of the invention are from about 0.1 μg to about 100 mg/Kg body weight, administered at intervals of 4-72 hours, for a period of 2 hours to 1 year, and/or any range or value therein, such as 0.0001-0.001, 0.001-0.01, 0.01-0.1, 0.1-1.0, 1.0-10, 5-10, 10-20, 20-50 and 50-100 mg/Kg, at intervals of 1-4, 4-10, 10-16, 16-24, 24-36, 36-48, 48-72 hours, for a period of 1-14, 14-28, or 30-44 days, or 1-24 weeks, or any range or value therein. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The composition may be intended for rectal administration, in the form, e.g., of a suppository which will melt in the rectum and release the drug. A typical suppository formulation will generally consist of active ingredient with a binding and/or lubricating agent such as a gelatine or cocoa butter or other low melting vegetable or synthetic wax or fat.

A typical formulation for intramuscular or intrathecal administration Will consist of a suspension or solution of active in an oil or solution of active ingredient in an oil, for example arachis oil or seasame oil. A typical formulation for intravenous or intrathecal administration will consist of sterile isotonic aqueous solution containing, for example active ingredient and dextrose or sodium chloride or a mixture of dextrose and sodium chloride.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as desscribed in PCT Patent No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues) Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

Combination Therapy

The compounds of the invention may be usefully combined with one or more other compounds of the invention or one or more other therapeutic agent or as any combination thereof, in the treatment of sodium channel-mediated diseases and conditions. For example, a compound of formula (I) may be administered simultaneously, sequentially or separately in combination with other therapeutic agents, including, but not limited to:
opiates analgesics, e.g. morphine, heroin, cocaine, oxymorphine, levorphanol, levallorphan, oxycodone, codeine, dihydrocodeine, propoxyphene, nalmefene, fentanyl, hydrocodone, hydromorphone, meripidine, methadone, nalorphine, naloxone, naltrexone, buprenorphine, butorphanol, nalbuphine and pentazocine;
non-opiate analgesics, e.g. acetomeniphen, salicylates (e.g. aspirin);
nonsteroidal antiinflammatory drugs (NSAIDs), e.g. ibuprofen, naproxen, fenoprofen, ketoprofen, celecoxib, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac;
anticonvulsants, e.g. carbamazepine, oxcarbazepine, lamotrigine, valproate, topiramate, gabapentin and pregabalin;
antidepressants such as tricyclic antidepressants, e.g. amitriptyline, clomipramine, despramine, imipramine and nortriptyline;
COX-2 selective inhibitors, e.g. celecoxib, rofecoxib, parecoxib, valdecoxib, deracoxib, etoricoxib, and lumiracoxib;
alpha-adrenergics, e.g. doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl)quinazoline;
barbiturate sedatives, e.g. amobarbital, aprobarbital, butabarbital, butabital, mephobarbital, metharbital, methohexital, pentobarbital, phenobartital, secobarbital, talbutal, theamylal and thiopental;
tachykinin (NK) antagonists, particularly an NK-3, NK-2 or NK-1 antagonist, e.g. (ccR,9R)-7-&lsqb; 3,5-bis(trifluoromethyl)benzyl&rsqb;-8,9,10,11-tetrahydro-9-methyl-5 (4-methylphenyl)-7H-&lsqb;1,4&rsqb;diazocino&lsqb;2,1-g&rsqb;&lsqb;1,7&rsqb;-naphthyridine-6-13-dione (TAK 637), 5-&lsqb;&lsqb;(2R,3S)-2-&lsqb;(1 R)-1-&lsqb;3,5-bis(trifluoromethyl)phenyl&rsqb;ethoxy-3-(4 fluorophenyl)-4-morpholinyl&rsqb;-methyl&rsqb;-1,2-dihydro-3H-1,2,4-triazol-3-one (MK 869), ap rep itant, lane p itant, dapitant and 3-&lsqb;&lsqb;2-methoxy-5 (trifluoromethoxy)phenyl&rsqb;-methylamino&rsqb;-2-phenylpiperidine (2S, 3S);
coal-tar analgesics, in particular paracetamol;
serotonin reuptake inhibitors, e.g. paroxetine, sertraline, norfluoxetine (fluoxetine desmethyl metabolite), metabolite demethylsertraline, '3 fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, trazodone and fluoxetine;
noradrenaline (norepinephrine) reuptake inhibitors, e.g. maprotiline, lofepramine, mirtazepine, oxaprotiline, fezolamine, tomoxetine, mianserin, buproprion, buproprion metabolite hydroxybuproprion, nomifensine and viloxazine (Vivalan®)), especially a selective noradrenaline reuptake inhibitor such as reboxetine, in particular (S,S)-reboxetine, and venlafaxine duloxetine neuroleptics sedative/anxiolytics;
dual serotonin-noradrenaline reuptake inhibitors, such as venlafaxine, venlafaxine metabolite O-desmethylvenlafaxine, clomipramine, clomipramine metabolite desmethylclomipramine, duloxetine, milnacipran and imipramine;
acetylcholinesterase inhibitors such as donepezil;
5-HT3 antagonists such as ondansetron;
metabotropic glutamate receptor (mGluR) antagonists;
local anaesthetic such as mexiletine and lidocaine;
corticosteroid such as dexamethasone;
antiarrhythimics, e.g. mexiletine and phenytoin;
muscarinic antagonists, e.g., tolterodine, propiverine, tropsium t chloride, darifenacin, solifenacin, temiverine and ipratropium;
cannabinoids;
vanilloid receptor agonists (e.g. resinferatoxin) or antagonists (e.g. capsazepine);
sedatives, e.g. glutethimide, meprobamate, methaqualone, and dichloralphenazone;
anxiolytics such as benzodiazepines,
antidepressants such as mirtazapine,
topical agents (e.g. lidocaine, capsacin and resiniferotoxin);
muscle relaxants such as benzodiazepines, baclofen, carisoprodol, chlorzoxazone, cyclobenzaprine, methocarbamol and orphrenadine;
anti-histamines or H1 antagonists;
NMDA receptor antagonists;
5-HT receptor agonists/antagonists;
PDEV inhibitors;
Tramadol®;
cholinergic (nicotinc) analgesics;
alpha-2-delta ligands;
prostaglandin E2 subtype antagonists;
leukotriene B4 antagonists;
5-lipoxygenase inhibitors; and
5-HT3 antagonists.

Sodium channel-mediated diseases and conditions that may be treated and/or prevented using such combinations include but not limited to, pain, central and peripherally mediated, acute, chronic, neuropathic as well as other diseases with associated pain and other central nervous disorders such as epilepsy, anxiety, depression and bipolar disease; or cardiovascular disorders such as arrhythmias, atrial fibrillation and ventricular fibrillation; neuromuscular disorders such as restless leg syndrome and muscle paralysis or tetanus; neuroprotection against stroke, neural trauma and multiple sclerosis; and channelopathies such as erythromyalgia and familial rectal pain syndrome.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

Kits-of-Parts

The present invention also provides kits that contain a pharmaceutical composition which includes one or more compounds of the above formulae. The kit also includes instructions for the use of the pharmaceutical composition for modulating the activity of ion channels, for the treatment of pain, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### PREPARATION OF THE COMPOUNDS OF THE INVENTION

The following Reaction Schemes illustrate methods to make compounds of this invention, i.e., compounds of formula (I): 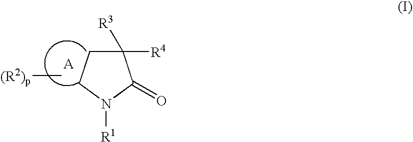
wherein 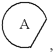
p, R¹, R², R³ and R⁴ are as defined herein, as a stereoisomer, enantiomer, tautomer thereof or mixtures thereof; or a pharmaceutically acceptable salt, solvate or prodrug thereof.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are known to one skilled in the art and as described herein.

The use of protecting groups is described in detail in Green, T. W. and P. G. M. Wuts, Protective Groups in Organic Synthesis (1999), 3rd Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

The following Reaction Schemes illustrate methods to make compounds of this invention. It is understood that one skilled in the art would be able to make these compounds by similar methods or by methods known to one skilled in the art. It is also understood that one skilled in the art would be able to make in a similar manner as described below other compounds of formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Smith, M. B. and J. March, Advanced Organic Chemistry: Reactions, Mechanisms, and Structure, 5th edition (Wiley, December 2000)) or prepared as described in this invention.

In the following Reaction Schemes, R¹, R², R⁴ and p are defined as in the Summary of the Invention unless specifically defined otherwise, and X is Cl or Br.

In general, the compounds of formula (I) of the invention where R³ is —OH can be synthesized following the general procedure as described in REACTION SCHEME 1.

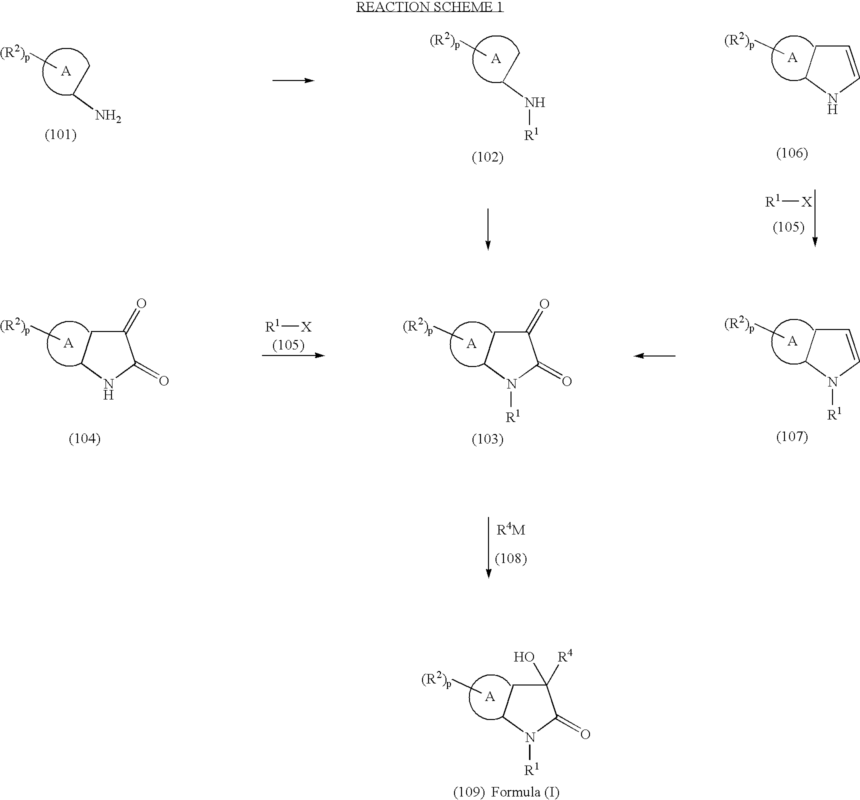

R¹ group can be introduced to an amino compound of formula (101) either by reductive amination, which is well-known to those skilled in the art, or formation of an amide by reacting with a corresponding acyl chloride followed by reduction, which is also well-known to those skilled in the art, to form a high order of amino compound of formula (102). Reaction of the compound of formula (102) with oxalyl chloride gives the compound of formula (103). Alternatively, the compound of formula (103) can be obtained by alkylation of the compound of formula (104) with the chloro or bromo compound of formula (105). Alternatively, alkylation of pyrrole-type compound of formula (106) with the chloro or bromo compound of formula (105) provides the compound of formula (107). Treatment of the compound of formula (107) with N-bromosuccinimide in a solvent such as, but not limited to, dimethylsulfoxide, affords the product of formula (103). The treatment of the compound of formula (103) with a nucleophile such as, but not limited to, a Grignard reagent or enolate of formula (108), affords the compound of formula (I) (109) of the invention where R³ is —OH.

In general, the compounds of formula (I) of the invention where R³ is —H can be synthesized following the general procedure as described below in REACTION SCHEME 2.

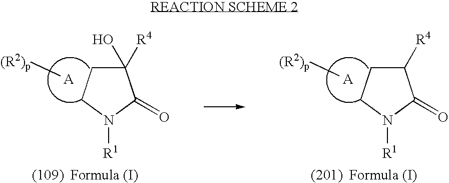

The compound of formula (201) can be obtained after the removal of the hydroxyl group of the heterocyclic compound of formula (109) by treating the compound with a silane such as triethylsilane. The compound of formula (201) can also be achieved by treating the compound of formula (109) with SOCl₂/NEt₃ followed by reduction with Zn dust.

In general, the compounds of formula (I) of the invention where R³ is —CH₂OH can be synthesized following the general procedure as described below in REACTION SCHEME 3.

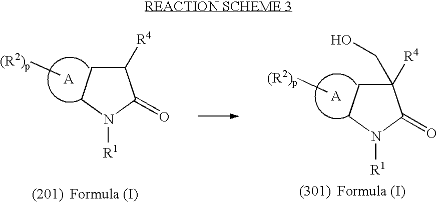

The compound (201) is treated with a silyl compound, such as, but not limited to, trimethylsilyl chloride, to generate the silyl ether intermediate, which is treated with ytterbium (III) trifluoromethanesulfonate and formaldehyde to afford the compound of formula (301). Alternatively, a compound of formula (301) can be obtained by treating the compound of formula (201) with a base, such as, but not limited to, LiOH, iPr₂NH, LDA, and subsequently reacting with formaldehyde.

In general, the compounds of formula (I) of the invention where R³ is fluoro can be synthesized following the general procedure as described below in REACTION SCHEME 4.

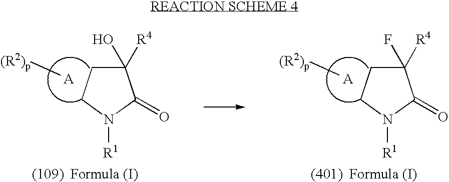

Treatment of the compound of formula (109) with a fluorinating reagent such as, but not limited to, diethylaminosulfur trifluoride (DAST), in a solvent such as, but not limited to, chloroform, provides the fluoro compound of the formula (I) (401).

In general, the compounds of formula (I) of the invention where R³ is —CN or —N(R⁵)R⁶ can be synthesized following the general procedure as described below in REACTION SCHEME 5.

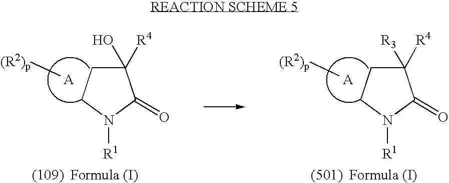

The hydroxyl group of the compound of the formula (109) can be converted to the corresponding chloro group by reacting with a chloride compound such as, but not limited to, thionyl chloride, in the presence of a base such as, but not limited to, diisopropylethylamine or triethylamine, in a solvent such as, but inot limited to, dichloromethane or chloroform. Treatment of the generated chloride compound with a nucleophile such as, but not limited to, sodium cyanide or benzylamine, in a solvent such as, but not limited to, tetrahedrofuran or dioxane, provides the compound of formula (I) (501).

The following specific Preparations (for the preparation of starting materials and intermediates) and Examples (for the preparation of the compounds of the invention) and the Biological Examples (for the assays used to demonstrate the utility of the compounds of the invention) are provided as a guide to assist in the practice of the invention, and are not intended as a limitation on the scope of the invention.

### Preparation 1

### Synthesis of 1-pentyl-1H-pyrrolo[1,2-b]pyrazole-2,3-dione

A. Synthesis of N-[(1E)-pentylidene]-1H-pyrrol-1-amine

A mixture of 1H-pyrrol-1-amine (4.0 g, 49.0 mmol), valeraldehyde (4.10 g, 49.0 mmol) and molecular sieves (4 Å) in ethanol (30.0 mL) was stirred at ambient temperature overnight. The reaction mixture was filtered and the filtrate was concentrated under reduced pressure to dryness to give the title compound (unstable): MS (ES+) m/z 151.2 (M+1).

B. Synthesis of N-pentyl-1H-pyrrol-1-amine

To a solution of N-[(1E)-pentylidene]-1H-pyrrol-1-amine in THF (100 mL) was added LiAlH₄ (3.80 g, 100 mmol) in small portions. The reaction mixture was stirred at ambient temperature for 20 h and quenched with the addition of saturated sodium sulfate solution dropwise. The mixture was filtered through celite, and the filtrate was concentrated under reduced pressure. The residue was subjected to column chromatography to afford the title compound (3.65 g, 51%): MS (ES+) m/z 153.2 (M+1).

C. Synthesis of 1-pentyl-1H-pyrrolo[1,2-b]pyrazole-2,3-dione

To a solution of N-pentyl-1H-pyrrol-1-amine (7.00 g, 46.0 mmol) in dichloroethane (200 mL) was added oxalyl chloride (7.50 g, 60.0 mmol) at −78° C. The reaction mixture was stirred at ambient temperature overnight and quenched with water. The organic layer was separated, dried over anhydrous sodium sulfate and filtered. The filtrate was concentrated under reduced pressure. The residue was subjected to column chromatography to afford the title compound (0.70 g, 7%): MS (ES+) m/z 229.3 (M+23).

### Preparation 2

### Synthesis of 6-pentyl-4H-thieno[2,3-b]pyrrole-4,5(6H)-dione

A. Synthesis of N-pentylthiophen-2-amine

A mixture of 2-iodothiophene (21.0 g, 100 mmol), n-pentylamine (13.5 g, 150 mmol), Cu metal (0.64 g), K₃PO₄ (42.4 9, 200 mmol) and water (3.60 g) in 2-(dimethylamino)ethanol (100 mL) was heated at 60° C. for 16 hours. The reaction mixture was poured into water and extracted with ether. The ether layer was separated, washed with brine, dried over anhydrous sodium sulfate and filtered. The filtrate was concentrated in vacuo to dryness to give the title compound (8.90 g, 53%): MS (ES+) m/z 170.3 (M+1).

B. Synthesis of 6-pentyl-4H-thieno[2,3-b]pyrrole-4,5(6H)-dione

A mixture of N-pentylthiophen-2-amine (8.90 g, 53.0 mmol) and oxalyl chloride (11.0 g, 87.0 mmol) in chloroform (200 mL) was heated at 60° C. for 5 hours. The reaction mixture was washed with water, brine, dried over anhydrous sodium sulfate and filtered. The filtrate was concentrated in vacuo to dryness. The residue was subjected to column chromatography to afford the title compound (0.90 g, 8%): MS (ES+) m/z 246.3 (M+23).

### Preparation 3

### Synthesis of 4-pentyl-4H-thieno[3,2-b]pyrrole-5,6-dione

A. Synthesis of N-3-thienylpentanamide

To a solution of thiophen-3-amine (Galvez, C., et al, J. Heterocycl. Chem. (1984), 21:393-5) (5.70 g, 57.0 mmol) and triethylamine (5.82 g, 58.0 mmol) in dichloromethane (100 mL) was added pentanoyl chloride (6.93 g, 57.0 mmol) dropwise at 0° C. The reaction mixture was stirred at ambient temperature overnight and quenched with water (50.0 mL). The organic layer was dried over anhydrous sodium sulfate and filtered. The filtrate was concentrated in vacuo to dryness to afford the title compound: MS (ES+) m/z 184.3 (M+1).

B. Synthesis of N-pentylthiophen-3-amine

To a solution of N-3-thienylpentanamide (13.4 g, 73.0 mmol) in THF (200 mL) was added LiAlH₄ (3.50 g, 100 mmol) at ambient temperature. The resulting mixture was stirred at ambient temperature for 16 h and at 60° C. for 1 h. After cooling down to ambient temperature, the reaction was quenched by the addition of saturated sodium sulfate dropwise until the color changed from green to white and diluted with THF (200 mL). The reaction mixture was filtered through celite and the filtrate was concentrated in vacuo to dryness. The residue was subjected to column chromatography to yield the title compound (9.70 g, 79%): MS (ES+) m/z 170.3 (M+1).

C. Synthesis of 4-pentyl-4H-thieno[3.2-b]pyrrole-5,6-dione

To a solution of N-pentylthiophen-3-amine (7.30 g, 4.30 mmol) in ether (50.0 mL) was added a solution of oxalyl chloride (6.00 mL, 42.0 mmol) in ether (50.0 mL) slowly at −10° C. The reaction mixture was stirred at ambient temperature for 3 h and quenched with cold water. The organic layer was separated, dried over anhydrous sodium sulfate and filtered. The filtrate was concentrated in vacuo to dryness. The residue was subjected to column chromatography to afford the title compound (5.10 g, 53%): MS (ES+) m/z 246.3 (M+23).

### Preparation 4

### Synthesis of 1-pentyl-1H-pyrrolo[2,3-b]pyridine-2,3-dione

A. Synthesis of 1-pentyl-1H-pyrrolo[2,3-b]pyridine

To a suspension of sodium hydride in anhydrous N,N-dimethylformamide (40.0 mL) was added 1H-pyrrolo[2,3-b]pyridine (5.00 g, 42.4 mmol) at 0° C. The reaction mixture was stirred for 0.5 h, followed by the addition of 1-bromopentane (9.25 g, 61.2 mmol). The reaction mixture was stirred at ambient temperature for 3.5 h, quenched with water (20.0 mL) and extracted with ethyl acetate (3×100 mL). The combined organic layers was washed with water (3×50.0 mL), dried over anhydrous sodium sulfate and filtered. The filtrate was concentrated in vacuo to dryness to give the title compound (8.00 g, 100%) as a pale yellow oil: ¹H NMR (300 MHz, CDCl₃) δ 8.29 (dd, 1H), 7.86 (d, 1H), 7.19 (d, 1H), 7.02-6.98 (m, 1H), 6.41 (d, 1H), 4.25 (t, 2H), 1.89-1.79 (m, 2H), 1.35-1.25 (m, 4H), 0.85 (t, 3H); ¹³C NMR (75 MHz, CDCl₃) δ 147.4, 142.6 128.6, 127.9, 120.6, 115.5, 99.2, 44.6, 30.1, 29.0, 22.4, 13.9.

B. Synthesis of 1-pentyl-1H-pyrrolo[2,3-b]pyridine-2,3-dione

A 2-neck round bottom flask (1 L) was charged with 1-pentyl-1H-pyrrolo[2,3-b]pyridine (17.4 g, 92.6 mmol) in anhydrous dimethylsulfoxide (300 mL) and bubbled with nitrogen. To the reaction solution was added N-bromosuccinimide (34.3 g, 193 mmol) in portion over 15 min at 0° C. The reaction mixture was heated at 60° C. for 6 h followed by at ambient temperature for 16 h. The reaction mixture was diluted with water (200 mL) and stirred for 0.5 h followed by extraction with ethyl acetate (3×200 mL). The combined organic layers was dried over anhydrous sodium sulfate and filtered. The filtrate was concentrated in vacuo to dryness to give the title compound as a yellow solid, which was crystallized from ether as an orange solid (14.6 g, 72%): ¹H NMR (300 MHz, CDCl₃) δ 8.41 (dd, 1H), 7.78 (dd, 1H), 7.03 (dd, 1H), 3.79 (t, 2H), 1.77-1.66 (m, 2H), 1.34-1.29 (m, 4H), 0.85 (t, 3H); ¹³C NMR (75 MHz, CDCl₃) δ 219.1, 182.2, 164.0, 158.2, 155.8, 132.8, 119.4, 112.0, 39.3, 28.9, 27.2, 22.3, 13.9.

### Preparation 5

### Synthesis of 1-pentyl-1H-pyrrolo[3,2-b]pyridine-2,3-dione

A. Synthesis of 1-pentyl-1H-pyrrolo[3,2-b]pyridine

Following the procedure as described in PREPARATION 4A, and making non-critical variations using 1H-pyrrolo[3,2-b]pyridine to replace 1H-pyrrolo[2,3-b]pyridine, the title compound was obtained (75%) as a yellow oil: ¹H NMR (300 MHz, CDCl₃) δ 8.39 (d, 1H), 7.56 (d, 1H), 7.25 (d, 1H), 7.05-7.01 (m, 1H), 6.63 (d, 1H), 4.05-3.99 (m, 2H), 1.79-1.72 (m, 2H), 1.31-1.45 (m, 4H), 0.81 (t, 3H); ¹³C NMR (75 MHz, CDCl₃) δ 146.8, 142.9, 131.0, 128.9, 116.5, 116.1, 102.0, 46.6, 30.0, 29.0, 22.2, 14.0; MS (ES+) m/z 189.3 (M+1).

B. Synthesis of 1-pentyl-1H-pyrrolo[3,2-b]pyridine-2,3-dione

Following the procedure as described in PREPARATION 48, and making non-critical variations using 1-pentyl-1H-pyrrolo[3,2-b]pyridine to replace 1-pentyl-1H-pyrrolo[2,3-b]pyridine, the title compound was obtained (44%) as a yellow solid: R_{f}=0.22 (ethyl acetate/hexane, 30%).

### Preparation 6

### Synthesis of 1-pentyl-1H-pyrrolo[3,2-c]pyridine-2,3-dione

Following the procedure as described in PREPARATION 4A, and making non-critical variations using 1H-pyrrolo[3,2-c]pyridine-2,3-dione (Rivalle, C., et al, J. Heterocycl. Chem. (1997), 34:441) to replace 1H-pyrrolo[2,3-b]pyridine, the title compound was obtained (36%): ¹H NMR (300 MHz, CDCl₃) δ 8.71-8.64 (m, 2H), 6.90 (d, 1H), 3.71 (t, 2H), 1.74-1.62 (m, 2H), 1.41-1.27 (m, 4H), 0.89 (t, 3H); MS (ES+) m/z 219.3 (M+1).

### EXAMPLE 1

### Synthesis of 3-(1,3-benzodioxol-5-yl)-3-hydroxy-1-pentyl-1H-pyrrolo[1,2-b]pyrazol-2(3H)-one

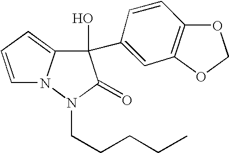

To a solution of 1-pentyl-1H-pyrrolo[1,2-b]pyrazole-2,3-dione (0.70 g, 3.40 mmol) in THF was added 3,4-(methylenedioxy)phenylmagnesium bromide (4.00 mL, 1.0 M solution in THF/toluene, 4.00 mmol) at 10° C. The reaction mixture was stirred at ambient temperature for two hours and quenched with saturated ammonium chloride solution. The organic layer was separated, dried over sodium sulfate and filtered. The filtrate was concentrated in vacuo to dryness. The residue was subjected to column chromatography to afford the title compound (0.09 g, 8%): ¹H NMR (300 MHz, CDCl₃) δ 7.00 (d, 1H), 6.89 (dd, 1H), 6.78 (dd, 1H), 6.73 (d, 1H), 6.28-6.18 (m, 2H), 5.93 (s, 2H), 3.89 (dt, 2H), 3.05 (br, 1H), 1.80-1.68 (m, 2H), 1.32 (dt, 4H), 0.86 (t, 3H); MS (ES+) m/z 351.3 (M+23).

### EXAMPLE 2

### Synthesis of 4-(1,3-benzodioxol-5-yl)-4-hydroxy-6-pentyl-4,6-dihydro-5H-thieno[2,3-b]pyrrol-5-one

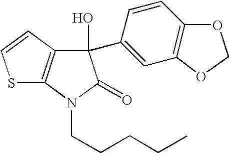

Following the procedure as described in Example 1, and making non-critical variations using 6-pentyl-4H-thieno[2,3-b]pyrrole-4,5(6H)-dione to replace 1-pentyl-1H-pyrrolo[1,2-b]pyrazole-2,3-dione, the title compound was obtained (32%): ¹H NMR (300 MHz, CDCl₃) δ 6.92-6.71 (m, 5H), 5.92 (s, 2H), 3.75-3.52 (m, 2H), 2.99 (br, 1H), 1.78-1.67 (m, 2H), 1.38-1.28 (m, 4H), 0.87 (t, 3H); ¹³C NMR (75 MHz, CDCl₃) δ 179.5, 147.9, 147.6, 146.9, 133.5, 128.1, 121.8, 119.1, 117.1, 108.1, 106.6, 101.2, 78.5, 42.7, 28.7, 27.3, 22.2, 13.9; MS (ES+) m/z 368.3 (M+23).

### EXAMPLE 3

### Synthesis of 6-(1,3-benzodioxol-5-yl)-6-hydroxy-4-pentyl-4,6-dihydro-5H-thieno[3,2-b]pyrrol-5-one

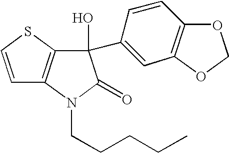

Following the procedure as described in Example 1, and making non-critical variations using 4-pentyl-4H-thieno[3,2-b]pyrrole-5,6-dione to replace 1-pentyl-1H-pyrrolo[1,2-b]pyrazole-2,3-dione, the title compound was obtained (21%): ¹H NMR (300 MHz, CDCl₃) δ 7.39 (d, 1H), 6.91-6.71 (m, 4H), 5.92 (s, 2H), 3.65 (m, 2H), 3.16 (br, 1H), 1.68 (m, 2H), 1.31 (m, 4H), 0.87 (t, 3H); ¹³C NMR (75 MHz, CDCl₃) δ 180.0, 147.8, 147.5, 146.1, 134.2, 129.5, 122.9, 119.1, 111.8, 108.0, 106.5, 101.2, 79.2, 41.9, 28.8, 27.8, 22.3, 14.0; MS (ES+) m/z 368.3 (M+23).

### EXAMPLE 4

### Synthesis of 3-hydroxy-3-(6-hydroxy-1,3-benzodioxol-5-yl)-1-pentyl-1,3-dihydro-2H-pyrrolo[2,3-b]pyridin-2-one

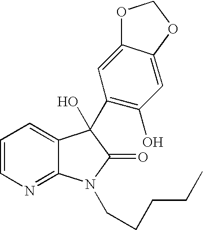

To a solution of 1,3-benzodioxol-5-ol in THF (40.0 mL) was added a solution of iso-propyl magnesium chloride (7.90 mL, 15.9 mmol, 2.0 M in THF) dropwise at 0° C. over 5 min. The reaction mixture was stirred for 30 min upon which time colorless precipitate formed. After the solvent was removed under reduced pressure, the residue was dissolved in anhydrous dichloromethane (40.0 mL) and cooled to 0° C. followed by the addition of a solution of 1-pentyl-1H-pyrrolo[2,3-b]pyridine-2,3-dione (1.84 g, 8.44 mmol) in dichloromethane (10.0 mL). The reaction mixture was stirred at ambient temperature for 16 h and quenched with saturated ammonium chloride solution (30.0 mL). The organic layer was separated and washed with water (3×25.0 mL), dried over anhydrous sodium sulfate and filtered. The filtrate was concentrated in vacuo to dryness. The residue was crystallized from ethyl acetate and ether to afford the title compound (2.20 g, 73%) as a beige solid: ¹H NMR (300 MHz, CDCl₃) δ 8.29 (dd, 1H), 7.74 (dd, 1H), 7.08 (dd, 1H), 6.60 (s, 1H), 6.24 (s, 1H), 5.87 (dd, 2H), 3.78 (d, 2H), 1.77-1.67 (m, 2H), 1.33-1.28 (m, 4H), 0.85 (d, 3H); ¹³C NMR (75 MHz, DMSO-d₆) δ 176.9, 157.7, 148.9, 147.3, 147.2, 139.7, 131.1, 127.7, 119.3, 118.3, 107.1, 101.1, 97.8, 74.6, 40.7, 29.0, 27.0, 22.3, 14.4; MS (ES+) m/z 357 (M+1).

### EXAMPLE 5

### Synthesis of 3-hydroxy-3-(6-hydroxy-1,3-benzodioxol-5-yl)-1-pentyl-1,3-dihydro-2H-pyrrolo[3,2-b]pyridin-2-one

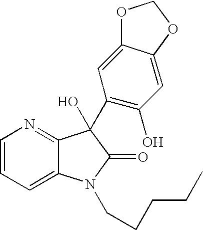

Following the procedure as described in EXAMPLE 4, and making non-critical variations using 1-pentyl-1H-pyrrolo[3,2-b]pyridine-2,3-dione to replace 1-pentyl-1H-pyrrolo[2,3-b]pyridine-2,3-dione, the title compound was obtained (71%) as a pale yellow solid: ¹H NMR (300 MHz, CDCl₃) δ 8.17 (d, 1H), 7.29-7.26 (m, 1H), 7.16 (d, 1H), 6.52 (s, 1H), 6.43 (s, 1H), 5.82 (d, 2H), 3.86-3.76 (m, 1H), 3.70-3.57 (m, 1H), 1.68-1.63 (m, 2H), 1.33-1.31 (m, 4H), 0.86 (t, 3H); ¹³C NMR (75 MHz, CDCl₃) δ 174.8, 153.3, 151.0, 149.0, 141.8, 141.0, 137.0, 124.8, 116.3, 115.3, 106.8, 101.9, 101.4, 77.5, 40.3, 28.9, 26.8, 22.2, 13.9; MS (ES+) m/z 357.5 (M+1), 339.5 (M−17).

### EXAMPLE 6

### Synthesis of 3-hydroxy-3-(6-hydroxy-1,3-benzodioxol-5-yl)-1-pentyl-1,3-dihydro-2H-pyrrolo[3,2-c]pyridin-2-one

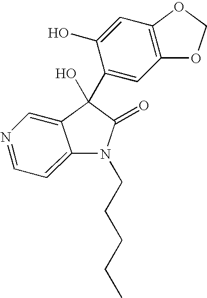

To a solution of 1,3-benzodioxol-5-ol (0.27 g, 1.90 mmol) in THF (10.0 mL) was added iso-propylmagnesium chloride.(0.97 mL, 2 M solution in THF, 1.90 mmol) slowly at 0° C. The mixture was allowed to stir at ambient temperature for 1 hour followed by the addition of 1-pentyl-1H-pyrrolo[3,2-c]pyridine-2,3-dione (0.21 g, 0.96 mmol). The resulting mixture was stirred at ambient temperature overnight, quenched with saturated ammonium chloride (20.0 mL). The mixture was extracted with ethyl acetate (3×50.0 mL). The combined organic layers was dried over anhydrous sodium sulfate and filtered. The filtrate was concentrated in vacuo. The residue was subjected to column chromatography (ethyl acetate/hexane, 1/2) to give the title compound (0.52 g, 40%) as a white solid: mp 193-195° C.; ¹H NMR (300 MHz, DMSO-d₆) δ 9.12 (s, 1H), 8.30 (d, 1H), 7.88 (s, 1H), 7.22 (s, 1H), 7.04 (d, 1H), 6.64 (s, 1H), 6.21 (s, 1H), 5.93-5.87 (m, 2H), 3.70-3.50 (m, 2H), 1.63-1.48 (m, 2H), 1.36-1.23 (m, 4H), 0.84 (t, 3H); ¹³C NMR (75 MHz, DMSO-d₆) δ 177.0, 151.4, 150.6, 148.5, 147.3, 143.4, 140.0, 128.6, 119.6, 107.1, 104.6, 101.2, 97.8, 73.9, 28.9, 26.8, 22.4, 14.4; MS (ES+) m/z 357.2 (M+1).

### EXAMPLE 7

### Synthesis of 6-hydroxy-6-(6-hydroxy-1,3-benzodioxol-5-yl)-4-pentyl-4,6-dihydro-5H-thieno[3,2-b]pyrrol-5-one

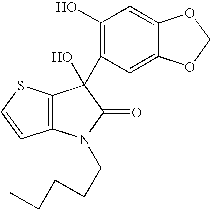

Following the procedure as described in EXAMPLE 4, and making non-critical variations using 4-pentyl-4H-thieno[3,2-b]pyrrole-5,6-dione to replace 1-pentyl-1H-pyrrolo[2,3-b]pyridine-2,3-dione, the title compound was obtained (26%) as a green solid: MS (ES+) m/z 384.4 (M+23).

### EXAMPLE 8

### Synthesis of 3-(6-hydroxy-1,3-benzodioxol-5-yl)-1-pentyl-1,3-dihydro-2H-pyrrolo[2,3-b]pyridin-2-one

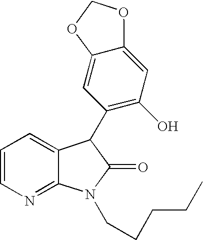

To a solution of 3-hydroxy-3-(6-hydroxy-1,3-benzodioxol-5-yl)-1-pentyl-1,3-dihydro-2H-pyrrolo[2,3-b]pyridin-2-one (4.00 g, 11.2 mmol) in anhydrous dichloromethane (80.0 mL) was added diisopropyl ethylamine (6.10 mL) and thionyl chloride (2.77 g, 23.5 mmol) under nitrogen at 0° C. The reaction mixture was stirred at 0° C. for 1 h and concentrated in vacuo to dryness. The residue was dissolved in THF/acetic acid (7:3, 100 mL) followed by the addition of Zn dust (3.08 g, 47.1 mmol) in one portion. The reaction mixture was stirred at ambient temperature for 16 h, filtered and the residue was washed with ethyl acetate (30.0 mL). The filtrate was concentrated in vacuo to dryness. The residue was dissolved in ethyl acetate (200 mL), washed with saturated ammonium chloride (3×50.0 mL), dried over anhydrous sodium sulfate and filtered. The filtrate was concentrated in vacuo to dryness. The residue was subjected to column chromatography to give the title compound (2.92 g, 76%) as a solid: ¹H NMR (300 MHz, CDCl₃) δ 8.64 (br, 1H), 8.26 (d, 1H), 7.52 (d, 1H), 7.05 (dd, 1H), 6.53 (s, 1H), 6.25 (s, 1H), 5.84 (d, 2H), 5.02 (s, 1H), 3.86-3.75 (m, 2H), 1.76-1.67 (m, 2H), 1.33-1.28 (m, 4H), 0.85 (t, 3H); ¹³C NMR (75 MHz, CDCl₃) δ 178.5, 157.4, 150.9, 147.8, 147.5, 141.6, 133.2, 121.7, 118.7, 114.1, 106.4, 101.2, 101.1, 46.5, 39.8, 28.9, 27.3, 22.3, 13.9; MS (ES+) m/z 341 (M+1).

### EXAMPLE 9

### Synthesis of 3-(6-hydroxy-1,3-benzodioxol-5-yl)-1-pentyl-1,3-dihydro-2H-pyrrolo[3,2-b]pyridin-2-one

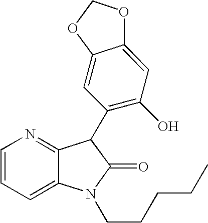

Following the procedure as described in EXAMPLE 8, and making non-critical variations using 3-hydroxy-3-(6-hydroxy-1,3-benzodioxol-5-yl)-1-pentyl-1,3-dihydro-2H-pyrrolo[3,2-b]pyridin-2-one to replace 3-hydroxy-3-(6-hydroxy-1,3-benzodioxol-5-yl)-1-pentyl-1,3-dihydro-2H-pyrrolo[2,3-b]pyridin-2-one, the title compound was obtained (50%): MS (ES+) m/z 341.1 (M+1).

### EXAMPLE 10

### Synthesis of 3-(6-hydroxy-1,3-benzodioxol-5-yl)-1-pentyl-1,3-dihydro-2H-pyrrolo[3,2-c]pyridin-2-one

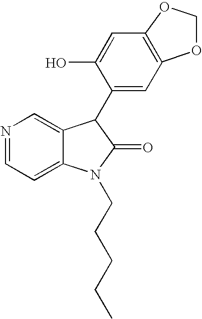

A mixture of 3-hydroxy-3-(6-hydroxy-1,3-benzodioxol-5-yl)-1-pentyl-1,3-dihydro-2H-pyrrolo[3,2-c]pyridin-2-one (0.15 g, 0.42 mmol), triethylsilane (1.60 mL, 10.0 mmol) and trifluroacetic acid (0.74 mL, 10.0 mmol) was stirred at ambient temperature overnight. The mixture was diluted with ethyl acetate (100 mL), washed with water, dried over anhydrous sodium sulfate and filtered. The filtrate was concentrated in vacuo. The residue was triturated with diethyl ether to give the title compound as a white solid (not stable, turned to red in the air): MS (ES+) m/z 341.4 (M+1).

### EXAMPLE 11

### Synthesis of 6-(6-hydroxy-1,3-benzodioxol-5-yl)-4-pentyl-4,6-dihydro-5H-thieno[3,2-b]pyrrol-5-one

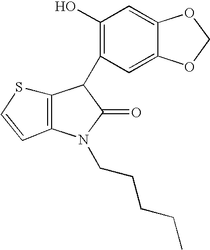

To a solution of 6-hydroxy-6-(6-hydroxy-1,3-benzodioxol-5-yl)-4-pentyl-4,6-dihydro-5H-thieno[3,2-b]pyrrol-5-one (1.71 g, 4.70 mmol) in CH₂Cl₂ (30.0 mL) were added trifluoroacetic acid (6.00 g, 52.6 mmol) and triethylsilane (5.00 g, 43.0 mmol) at 0° C. The reaction mixture was stirred at ambient temperature for 16 hours and diluted with CH₂Cl₂ (50.0 mL). The mixture was washed with water (2×50.0 mL), dried over Na₂SO₄ and filtered. The filtrate was evaporated under reduced pressure. The residue was subjected to column chromatography to give the title compound (0.80 g, 49%) as a green solid: MS (ES+) m/z 346.4 (M+1).

### EXAMPLE 12

### Synthesis of 3-(6-hydroxy-1,3-benzodioxol-5-yl)-3-(hydroxymethyl)-1-pentyl-1,3-dihydro-2H-pyrrolo[2,3-b]pyridin-2-one

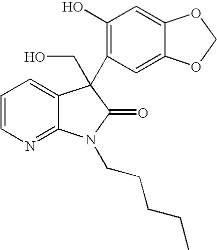

To a solution of 3-(6-hydroxy-1,3-benzodioxol-5-yl)-1-pentyl-1,3-dihydro-2H-pyrrolo[2,3-b]pyridin-2-one (2.75 g, 8.08 mmol) in anhydrous dichloromethane (40.0 mL) were added triethylamine (4.91 g, 48.5 mmol) and chlorotrimethylsilane (3.51 g, 32.3 mmol) under nitrogen at 0° C. The reaction mixture was stirred at 0° C. for 2 h and diluted with anhydrous dichloromethane (50.0 mL). The organic layer was washed with water (2×25.0 mL), dried over magnesium sulfate and filtered. The filtrate was concentrated in vacuo to dryness. The gummy brown residue was dissolved in THF (40.0 mL) followed by the addition of formaldehyde solution (2.20 mL, 80.8 mmol, 37 wt % in water) and ytterbium (III) trifluoromethanesulfonate (1.25 g, 2.02 mmol). The reaction mixture was stirred at ambient temperature for 36 h and diluted with dichloromethane (100 mL). The organic layer was washed with saturated NaHCO₃ (50.0 mL), saturated ammonium chloride (50.0 mL) and water (50.0 mL), dried over anhydrous sodium sulfate and filtered. The filtrate was concentrated in vacuo to dryness to afford the title compound (2.85 g, 98%): ¹H NMR (300 MHz, CDCl₃) δ 10.02 (s, 1H), 8.29 (dd, 1H), 7.72 (dd, 1H), 7.13 (dd, 1H), 6.55 (s, 1H), 6.46 (s, 1H), 5.86 (dd, 2H), 4.37 (dd, 2H), 3.77-3.84 (m, 2H), 3.25 (br, 1H), 1.63-1.77 (m, 2H), 1.36-1.22 (m, 4H), 0.85 (t, 3H); ¹³C NMR (75 MHz, CDCl₃) δ 179.9, 156.6, 152.3, 148.4, 147.5, 141.5, 133.8, 124.3, 118.7, 111.3, 107.9, 101.9, 101.4, 64.3, 59.1, 39.9, 31.6, 27.2, 22.3, 13.9; MS (ES+) m/z 371.1 (M+1).

### EXAMPLE 13

### Synthesis of 3-(6-hydroxy-1,3-benzodioxol-5-yl)-3-(hydroxymethyl)-1-pentyl-1,3-dihydro-2H-pyrrolo[3,2-b]pyridin-2-one

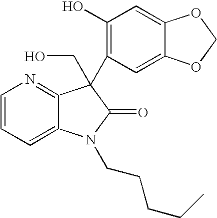

To a solution of 3-(6-hydroxy-1,3-benzodioxol-5-yl)-1-pentyl-1,3-dihydro-2H-pyrrolo[3,2-b]pyridin-2-one (1.60 g, 4.70 mmol) in anhydrous tetrahydrofuran (30.0 mL) was added a solution of pre-prepared lithium diisopropylamide (10.3 mmol) in anhydrous tetrahydrofuran (30.0 mL) at −78° C. The reaction mixture was stirred at −78° C. for 0.5 h followed by the addiotion of para-formaldehyde (0.85 g, 28.2 mmol) in one portion. The reaction was stirred at −78° C. for 2 h and quenched with saturated ammonium chloride (20.0 mL). After the organic solvent was removed under reduced pressure, the residue was diluted with ethyl acetate (50.0 mL). The organic layer was washed with brine (30.0 mL), dried over anhydrous sodium sulfate and filtered. The filtrate was concentrated in vacuo to dryness to give the title compound (1.95 g, 100%): ¹H NMR (300 MHz, CDCl₃) δ 8.22 (dd, 1H), 7.22-7.12 (m, 2H), 6.51 (s, 1H), 6.06 (s, 1H), 5.83 (d, 2H), 4.89 (s, 2H), 3.83-3.61 (m, 2H), 1.75-1.61 (m, 2H), 1.39-1.29 (m, 4H), 0.89 (t, 3H).

### EXAMPLE 14

### Synthesis of 3-(6-hydroxy-1,3-benzodioxol-5-yl)-3-(hydroxymethyl)-1-pentyl-1,3-dihydro-2H-pyrrolo[3,2-c]pyridin-2-one

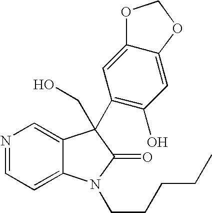

Following the procedure described in EXAMPLE 13, and making non-critical variations using 3-(6-hydroxy-1,3-benzodioxol-5-yl)-1-pentyl-1,3-dihydro-2H-pyrrolo[3,2-c]pyridin-2-one to replace 3-(6-hydroxy-1,3-benzodioxol-5-yl)-1-pentyl-1,3-dihydro-2H-pyrrolo[3,2-b]pyridin-2-one, the title compound was obtained: MS (ES+) m/z 371.4(M+1).

### EXAMPLE 15

### Synthesis of 6-(6-hydroxy-1,3-benzodioxol-5-yl)-6-(hydroxymethyl)-4-pentyl-4,6-dihydro-5H-thieno[3,2-b]pyrrol-5-one

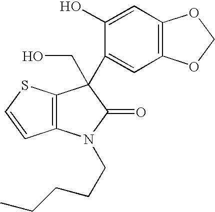

Following the procedure as described in EXAMPLE 12, and making non-critical variations using 6-(6-hydroxy-1,3-benzodioxol-5-yl)-4-pentyl-4,6-dihydro-5H-thieno[3,2-b]pyrrol-5-one to replace 3-(6-hydroxy-1,3-benzodioxol-5-yl)-1-pentyl-1,3-dihydro-2H-pyrrolo[2,3-b]pyridin-2-one, the title compound was obtained (10%): MS (ES+) m/z 376.1(M+1), 398.5 (M+23).

### EXAMPLE 16

### Synthesis of 3-(1,3-benzodioxol-5-yl)-3-hydroxy-1-pentyl-1,3-dihydro-2H-pyrrolo[2,3-b]pyridin-2-one

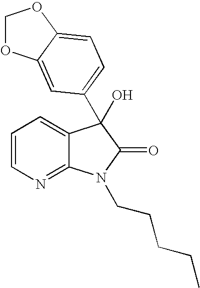

To a solution of 1-pentyl-1H-pyrrolo[2,3-b]pyridine-2,3-dione (0.32 g, 1.45 mmol) in anhydrous THF (20.0 mL) was added dropwise a solution of (3,4-methylenedioxy)phenyl bromide (2.20 mL, 1.0 M solution in THF/toluene, 2.17 mmol) at −78° C. under nitrogen. The reaction mixture was stirred at ambient temperature overnight and quenched with saturated NH₄Cl solution (15.0 mL). The mixture was concentrated in vacuo. The aqueous residue was extracted with ethyl acetate, dried over Na₂SO₄ and filtered. The filtrate was concentrated in vacuo to dryness The residue was subjected to column chromatography to give the title compound (0.46 g, 93%): mp 104-105° C.; ¹H NMR (300 MHz, CDCl₃) δ 8.18 (dd, 1H), 7.48 (dd, 1H), 6.93 (dd, 1H), 6.89 (d, 1H), 6.77 (dd, 1H), 6.71 (d, 1H), 5.92 (s, 2H), 4.04 (br, 1H), 3.78 (dt, 2H), 1.77-1.67 (m, 2H), 1.36-1.27 (m, 4H), 0.86 (t, 3H); ¹³C NMR (75 MHz, CDCl₃) δ 177.3, 156.7, 148.6, 148.1, 147.9, 133.3, 132.3, 126.3, 118.8, 118.8, 108.3, 106.1, 101.3, 77.2, 39.5, 29.0, 27.3, 22.3, 14.0; MS (ES+) m/z 341 (M+1).

### EXAMPLE 17

### Synthesis of 3-(1,3-benzodioxol-5-yl)-1-pentyl-1,3-dihydro-2H-pyrrolo[2,3-b]pyridin-2-one

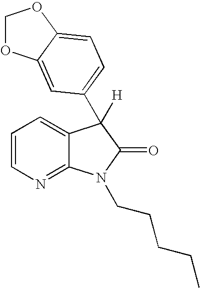

Following the procedure as described in EXAMPLE 8, and making non-critical variations using 3-(1,3-benzodioxol-5-yl)-3-hydroxy-1-pentyl-1,3-dihydro-2H-pyrrolo[2,3-b]pyridin-2-one to replace 3-hydroxy-3-(6-hydroxy-1,3-benzodioxol-5-yl)-1-pentyl-1,3-dihydro-2H-pyrrolo[2,3-b]pyridin-2-one, the title compound was obtained (75%): mp 75-77° C.; ¹H NMR (300 MHz, CDCl₃) δ 8.19 (d, 1H), 7.36 (d, 1H), 6.92 (dd, 1H), 6.75 (d, 1H), 6.64 (dd, 1H), 6.57 (d, 1H), 5.90 (s, 2H), 4.48 (s, 1H), 3.85-3.76 (m, 2H), 1.77-1.67 (m, 2H), 1.36-1.27 (m, 4H), 0.85 (t, 3H); ¹³C NMR (75 MHz, CDCl₃) δ 175.4, 157.6, 148.2, 147.4, 132.2, 129.1, 123.6, 121.8, 118.2, 108.7, 108.5, 101.2, 50.9 39.5, 29.0, 27.4, 22.4, 14.0; MS (ES+) m/z 326 (M+1).

### EXAMPLE 18

### Synthesis of 3-hydroxy-3-[2-oxo-2-(2-thienyl)ethyl]-1-pentyl-1,3-dihydro-2H-pyrrolo[2,3-b]pyridin-2-one

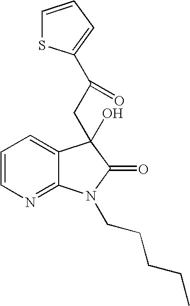

To a mixture of 1-pentyl-1H-pyrrolo[2,3-b]pyridine-2,3-dione (0.62 g, 2.82 mmol) in ethanol (12.0 mL) was added diisopropylethylamine (0.10 mL) and 1-thiophen-2-ylethanone (0.53 g, 4.23 mmol) at ambient temperature. The yellow reaction mixture was heated to reflux for 2 h, cooled to ambient temperature and kept stirring for 17 h upon which time precipitate was formed. The solid was collected by filtration, washed with methanol and ether to afford the title compound (0.63 g, 64%) as a colorless solid: ¹H NMR (300 MHz, CDCl₃) δ 8.18 (dd, 1H), 7.68-7.61 (m, 3H), 7.08 (dd, 1H), 6.89 (dd, 1H), 4.89 (s, 1H), 3.78-3.71 (m, 3H), 3.43 (d, 1H), 1.75-1.65 (m, 2H), 1.36-1.28 (m, 4H), 0.85 (t, 3H); ¹³C NMR (75 MHz, CDCl₃) δ 190.2, 176.0, 156.9, 148.5, 143.3, 135.1, 133.2, 131.9, 128.4, 124.5, 118.5, 73.9, 44.9, 39.5, 29.0, 27.2. 22.4, 14.0; MS (ES+) m/z 345 (M+1).

### EXAMPLE 19

### Synthesis of 3-[2-(2-furyl)-2-oxoethyl]-3-hydroxy-1-pentyl-1,3-dihydro-2H-pyrrolo[2,3-b]pyridin-2-one

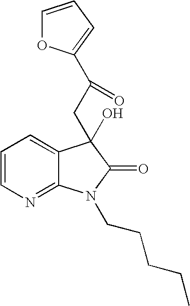

Following the procedure as described in EXAMPLE 18, making non-critical variations using 1-furan-2-ylethanone to replace 1-thiophen-2-ylethanone, the title compound was obtained (71%) as a colorless solid: ¹H NMR (300 MHz, CDCl₃) δ 8.14 (dd, 1H), 7.60 (dd, 1H), 7.54 (d, 1H), 7.18 (d, 1H), 6.89 (dd, 1H), 6.50 (dd, 1H), 4.82 (s, 1H), 3.74 (t, 2H), 3.49 (ABq, 2H), 1.75-1.65 (m, 2H), 1.34-1.28 (m, 4H), 0.84 (t, 3H); ¹³C NMR (75 MHz, CDCl₃) δ 186.1, 176.1, 156.9, 152.0, 148.5, 147.3, 131.9, 124.4, 118.5, 112.7, 73.9, 44.0, 39.4, 29.0, 27.1, 22.3, 14.0; MS (ES+) m/z 330 (M+2).

### EXAMPLE 20

### Synthesis of 3-(1,3-benzodioxol-5-yl)-3-fluoro-1-pentyl-1,3-dihydro-2H-pyrrolo[2,3-b]pyridin-2-one

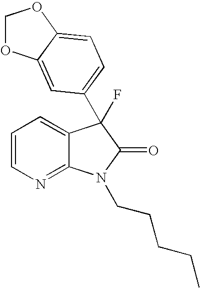

A solution of 3-(1,3-benzodioxol-5-yl)-3-hydroxy-1-pentyl-1,3-dihydro-2H-pyrrolo[2,3-b]pyridin-2-one (0.26 g, 0.76 mmol) in anhydrous chloroform (2.00 mL) was added dropwise to a solution diethylaminosulfur trifluoride (DAST) (0.18 g, 1.14 mmol) in anhydrous chloroform (7.00 mL) over 45 min under nitrogen at 0° C. The yellow reaction mixture was stirred at 0° C. for 4 h and diluted with ether (10.0 mL). The mixture was washed with water (2×5.00 mL), dried over anhydrous sodium sulfate and filtered. The filtrate was concentrated in vacuo to dryness. The residue was subjected to column chromatography to afford the title compound (0.17 g, 64%):\¹H NMR (300 MHz, CDCl₃) δ 8.31 (dt, 1H), 7.67 (dt, 1H), 7.09 (dd, 1H), 6.96 (d, 1H), 6.82 (d, 1H), 6.77-6.73 (m, 1H), 5.98 (d, 2H), 3.73 (t, 2H), 1.73-1.63 (m, 2H), 1.35-1.19 (m, 4H), 0.83 (t, 3H); ¹³C NMR (75 MHz, CDCl₃) δ 171.9, 171.6, 157.7, 157.6, 150.3, 150.3, 148.9 148.8, 148.3, 133.7 128.9 128.5, 121.2, 120.9, 119.8, 119.8, 119.1, 119.1, 117.3, 108.1, 106.3, 106.2, 102.0, 39.1, 28.6, 26.8, 22.0, 13.2; MS (ES+) m/z 343 (M+1), 323 (M−F).

### EXAMPLE 21

### Synthesis of 3-(1,3-benzodioxol-5-yl)-2-oxo-1-pentyl-2,3-dihydro-1H-pyrrolo[2,3-b]pyridine-3-carbonitrile

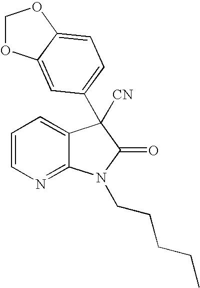

To a solution of 3-(1,3-benzodioxol-5-yl)-3-hydroxy-1-pentyl-1,3-dihydro-2H-pyrrolo[2,3-b]pyridin-2-one (0.68 g, 2.00 mmol) in anhydrous dichloromethane (20.0 mL) was added diisopropylethylamine (0.78 g, 6.00 mmol) followed by the addition of thionyl chloride (0.47 g, 4.00 mmol) at 0° C. The reaction mixture was stirred for 0.5 h and concentrated under reduced pressure. The gummy residue was dissolved in anhydrous tetrahydrofuran (20.0 mL) followed by the addition of sodium cyanide (0.20 g, 4.00 mmol). The reaction mixture was stirred at ambient temperature for 16 h, diluted with water (20.0 mL) and extracted with ethyl acetate (3×50.0 mL). The combined organic layers was washed with water (20.0 mL), saturated ammonium chloride (30.0 mL), and brine (20.0 mL). The organic layer was dried over anhydrous sodium sulfate and filtered. The filtrate was concentrated in vacuo to dryness. The residue was subjected to column chromatography to afford the title compound (0.46 g, 65%): MS (ES+) m/z 350 (M+1).

### EXAMPLE 22

### Synthesis of 3-(1,3-benzodioxol-5-yl)-3-(benzylamino)-1-pentyl-1,3-dihydro-2H-pyrrolo[2,3-b]pyridin-2-one

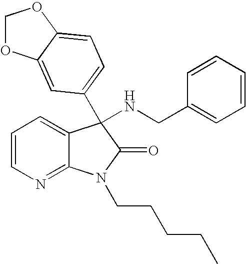

To a solution of 3-(1,3-benzodioxol-5-yl)-3-hydroxy-1-pentyl-1,3-dihydro-2H-pyrrolo[2,3-b]pyridin-2-one (0.68 g, 2.00 mmol) in anhydrous dichloromethane (20.0 mL) was added diisopropylethylamine (0.78 g, 6.00 mmol) and thionyl chloride (0.47 g, 4.00 mmol) at 0° C. The reaction mixture was stirred for 0.5 h, and concentrated under reduced pressure. The gummy residue was dissolved in anhydrous dioxane (20.0 mL) followed by the addition of benzylamine (0.43 g, 4.00 mmol). The reaction mixture was heated at reflux for 16 h, cooled to ambient temperature, diluted with water (20.0 mL) and extracted with ethyl acetate (3×50.0 mL). The combined organic layers was washed with water (20.0 mL), saturated ammonium chloride (30.0 mL), and brine (20.0 mL). The organic layer was dried over anhydrous sodium sulfate and filtered. The filtrate was concentrated in vacuo to dryness. The residue was subjected to column chromatography to afford the title compound (0.63 g, 73%) as a gummy material: MS (ES+) m/z 430 (M+1).

### Biological Assays

Various techniques are known in the art for testing the activity of compounds of the invention. In order that the invention described herein may be more fully understood, the following biological assays are set forth. It should be understood that these examples are for illustrative purposes only and are not to be construed as limiting this invention in any manner.

### BIOLOGICAL EXAMPLE 1

### Guanidine Influx Assay (In Vitro Assay)

This example describes an in vitro assay for testing and profiling test agents against human or rat sodium channels stably expressed in cells of either an endogenous or recombinant origin. The assay is also useful for determining the IC-50 of a sodium channel blocking compound. The assay is based on the guanidine flux assay described by Reddy, N. L., et al., J. Med. Chem. (1998), 41(17):3298-302.

The guanidine influx assay is a radiotracer flux assay used to determine ion flux activity of sodium channels in a high-throughput microplate-based format. The assay uses ¹⁴C-guanidine hydrochloride in combination with various known sodium channel modulators, to assay the potency of test agents. Potency is determined by an IC-50 calculation. Selectivity is determined by comparing potency of the compound for the channel of interest to its potency against other sodium channels (also called ‘selectivity profiling’).

Each of the test agents is assayed against cells that express the channels of interest. Voltage gated sodium channels are either TTX sensitive or insensitive. This property is useful when evaluating the activities of a channel of interest when it resides in a mixed population with other sodium channels. The following Table 1 summarizes cell lines useful in screening for a certain channel activity in the presence or absence of TTX.

TABLE 1

| CELL LINE | mRNA Expression | Functional Characterization |
|---|---|---|
| CHO-K1 (Chinese | Naᵥ1.4 expression has been | The 18-20-fold increase in [¹⁴C] |
| Hamster Ovary; | shown by RT-PCR | Guanidine influx was completely |
| recommended | No other Naᵥ expression has | blocked using TTX. (Naᵥ1.4 is a |
| host cell line) | been detected | TTX sensitive channel) |
| ATTC accession | | |
| number CCL-61 | | |
| L6 (rat myoblast | Expression of Nav1.4 and 1.5 | The 10-15 fold increase in [¹⁴C] |
| cell) ATTC | | Guanidine influx was only |
| Number CRL-1458 | | partially blocked by TTX (Naᵥ1.5 |
| | | is TTX resistant |
| SH-SY5Y (Human | Published Expression of | The 10-16-fold increase in [¹⁴C] |
| neuroblastoma) | Naᵥ1.9 and Naᵥ1.7 (Blum et | Guanidine influx above |
| ATTC Number | al) | background. |
| CRL-2266 | | was partially blocked by TTX |
| | | (Naᵥ1.9 is TTX resistant |
| SK-N-BE2C (a | Expression of NaV1.8 | Stimulation of BE2C cells with |
| human | | pyrethroids results in a 6 fold |
| neuroblastoma cell | | increase in [¹⁴C] Guanidine influx |
| line ATCC Number | | above background. |
| CRL-2268) | | TTX partially blocked influx |
| | | (NaV1.8 is TTX resistant) |
| PC12 (rat | Expression of Naᵥ1.2 | The 8-12-fold increase in [¹⁴C] |
| pheochromocytoma) | expression | Guanidine influx was completely |
| ATTC Number | | blocked using TTX. (Naᵥ1.2 is a |
| CRL-1721 | | TTX sensitive channel) |

It is also possible to employ recombinant cells expressing these sodium channels. Cloning and propagation of recombinant cells are known to those skilled in the art (see, for example, Klugbauer, N, et al., EMBO J. (1995), 14(6):1084-90; and Lossin, C., et al., Neuron (2002), 34:877-884).

Cells expressing the channel of interest are grown according to the supplier or in the case of a recombinant cell in the presence of selective growth media such as G418 (Gibco/Invitrogen) The cells are disassociated from the culture dishes with an enzymatic solution (1×) Trypsin/EDTA (Gibco/Invitrogen) and analyzed for density and viability using haemocytometer (Neubauer). Disassociated cells are washed and resuspended in their culture media then plated into Scintiplates (Beckman Coulter Inc.) (approximately 100,000 cells/well) and incubated at 37° C./5% CO_{2.} for 20-24 hours. After an extensive wash with Low sodium HEPES-buffered saline solution (LNHBSS) (150 mM Choline Chloride, 20 nM HEPES (Sigma), 1 mM Calcium Chloride, 5 mM Potassium Chloride, 1 mM Magnesium Chloride, 10 mM Glucose) agents diluted with LNHBSS are added to each well. (Varying concentrations of test agent may be used). The activation/radiolabel mixture contains aconitine (Sigma), and ¹⁴C-guanidine hydrochloride (ARC).

After loading the cells with test agent and activation/radiolabel mixture, the Scintiplates are incubated at ambient temperature. Following the incubation, the Scintplates are extensively washed with LNHBSS supplemented with guanidine (Sigma). The Scintiplates are dried and then counted using a Wallac MicroBeta TriLux (Perkin-Elmer Life Sciences). The ability of the test agent to block sodium channel activity is determined by comparing the amount of ¹⁴C-guanidine present inside the cells expressing the different sodium channels. Based on this data, a variety of calculations, as set out elsewhere in this specification, may be used to determine whether a test agent is selective for a particular sodium channel.

IC-50 value of a test agent for a specific sodium channel may be determined using the above general method. IC-50 may be determined using a 3, 8, 10, 12 or 16 point curve in duplicate or triplicate with a starting concentration of 1, 5 or 10 μM diluted serially with a final concentration reaching the sub-nanomolar, nanomolar and low micromolar ranges. Typically the mid-point concentration of test agent is set at 1 μM, and sequential concentrations of half dilutions greater or smaller are applied (e.g. 0.5 μM; 5 μM and 0.25 μM; 10 μM and 0.125 μM; 20 μM etc.). The IC-50 curve is calculated using the 4 Parameter Logistic Model or Sigmoidal Dose-Response Model formula (fit=(A+((B−A)/(1+((C/x)ˆD)))).

The fold selectivity, factor of selectivity or multiple of selectivity, is calculated by dividing the IC-50 value of the test sodium channel by the reference sodium channel, for example, Naᵥ1.5.

### BIOLOGICAL EXAMPLE 2

### Electrophysiological Assay (In Vitro Assay)

Cells expressing the channel of interest were cultured in DMEM growth media (Gibco) with 0.5 mg/mL G418, +/−1% PSG, and 10% heat-inactivated fetal bovine serum at 37C° and 5% CO₂. For electrophysiological recordings, cells were plated on 10 mm dishes.

Whole cell recordings were examined by established methods of whole cell voltage clamp (Bean et al., op. cit.) using an Axopatch 200B amplifier and Clampex software (Axon Instruments, Union City, Calif.). All experiments were performed at ambient temperature. Electrodes were fire-polished to resistances of 2-4 Mohm\tilde{s} Voltage errors and capacitance artifacts were minimized by series resistance compensation and capacitance compensation, respectively. Data were acquired at 40 kHz and filtered at 5 kHz. The external (bath) solution consisted of: NaCl (140 mM), KCl (5 mM), CaCl₂ (2 mM), MgCl₂ (1 mM), HEPES (10 mM) at pH 7.4. The internal (pipette) solution consisted of (in mM): NaCl (5), CaCl₂ (0.1) MgCl₂ (2), CsCl (10), CsF (120), HEPES (10), EGTA (10), at pH 7.2.

To estimate the steady-state affinity of compounds for the resting and inactivated state of the channel (Kᵣ and Kᵢ, respectively), 12.5 ms test pulses to depolarizing voltages from −60 to +90 mV from a holding potential of —110 mV was used to construct current-voltage relationships (I-V curves). A voltage near the peak of the IV-curve (−30 to 0 mV) was used as the test pulse throughout the remainder of the experiment. Steady-state inactivation (availability) curves were then constructed by measuring the current activated during a 8.75 ms test pulse following 1 second conditioning pulses to potentials ranging from −110 to −10 mV. To monitor channels at steady-state, a single “diary” protocol with a holding potential of −110 mV was created to record the resting state current (10 ms test pulse), the current after fast inactivation (5 ms pre-pulse of −80 to −50 mV followed by a 10 ms test pulse), and the current during various holding potentials (35 ms ramp to test pulse levels). Compounds were applied during the “diary” protocol and the block was monitored at 15 s intervals.

After the compounds equilibrated, the voltage-dependence of the steady-state inactivation in the presence of the compound was determined. Compounds that block the resting state of the channel decreased the current elicited during test pulses from all holding potentials, whereas compounds that primarily blocked the inactivated state decreased the current elicited during test pulses at more depolarized potentials. The currents at the resting state (Iᵣₑₛₜ) and the currents during the inactivated state (I_{inactivated}) were used to calculate steady-state affinity of compounds. Based on the Michaelis-Menton model of inhibition, the Kᵣ and Kᵢ was calculated as the concentration of compound needed to cause 50% inhibition of the Iᵣₑₛₜ or the I_{inactivated}, respectively.

$$\% inhibition = \frac{{V}_{max} * {[ Drug ]}^{h}}{{[ Drug ]}^{h} + {K}_{m}^{h}}$$

Vₘₐₓ is the rate of inhibition, h is the Hill coefficient (for interacting sites), Kₘ is Michaelis-Menten constant, and [Drug] is the concentration of the test compound. At 50% inhibition (½Vₘₐₓ) of the Iᵣₑₛₜ or I_{inactivated,} the drug concentration is numerically equal to Kₘ and approximates the Kᵣ and K_{i,} respectively.

### BIOLOGICAL EXAMPLE 3

### Analgesia Induced by Sodium Channel Blockers

Heat Induced Tail Flick Latency Test

In this test, the analgesia effect produced by administering a compound of the invention was observed through heat-induced tail-flick in mice. The test includes a heat source consisting of a projector lamp with a light beam focused and directed to a point on the tail of a mouse being tested. The tail-flick latencies, which were assessed prior to drug treatment, and in response to a noxious heat stimulus, i.e., the response time from applying radiant heat on the dorsal surface of the tail to the occurrence of tail flick, were measured and recorded at 40, 80, 120, and 160 minutes.

For the first part of this study, 65 animals underwent assessment of baseline tail flick latency once a day over two consecutive days. These animals were then randomly assigned to one of the 11 different treatment groups including a vehicle control, a morphine control, and 9 compounds at 30 mg/Kg were administered intramuscularly. Following dose administration, the animals were closely monitored for signs of toxicity including tremor or seizure, hyperactivity, shallow, rapid or depressed breathing and failure to groom. The optimal incubation time for each compound was determined via regression analysis. The analgesic activity of the test compounds was expressed as a percentage of the maximum possible effect (% MPE) and was calculated using the following formula:

$$\% MPE = \frac{Postdrug latency - Predrug latency}{Cut \text{-} off time ( 10 s ) - Predrug latency} \times 100 \%$$

where:

Postdrug latency=the latency time for each individual animal taken before the tail is removed (flicked) from the heat source after receiving drug.

Predrug latency=the latency time for each individual animal taken before the tail is flicked from the heat source prior to receiving drug.

Cut-off time (10 s)=is the maximum exposure to the heat source.

Acute Pain (Formalin Test)

The formalin test is used as an animal model of acute pain. In the formalin test, animals were briefly habituated to the plexiglass test chamber on the day prior to experimental day for 20 minutes. On the test day, animals were randomly injected with the test articles. At 30 minutes after drug administration, 50 μL of 10% formalin was injected subcutaneously into the plantar surface of the left hind paw of the rats. Video data acquisition began immediately after formalin administration, for duration of 90 minutes.

The images were captured using the Actimetrix Limelight software which stores files under the *.llii extension, and then converts it into the MPEG-4 coding. The videos are then analyzed using behaviour analysis software “The Observer 5.1”, (Version 5.0, Noldus Information Technology, Wageningen, The Netherlands). The video analysis was done by watching the animal behaviour and scoring each according to type, and defining the length of the behaviour (Dubuisson and Dennis, 1977). Scored behaviours include: (1) normal behaviour, (2) putting no weight on the paw, (3) raising the paw, (4) licking/biting or scratching the paw. Elevation, favoring, or excessive licking, biting and scratching of the injected paw indicate a pain response. Analgesic response or protection from compounds is indicated if both paws are resting on the floor with no obvious favoring, excessive licking, biting or scratching of the injected paw.

Analysis of the formalin test data is done according to two factors: (1) Percent Maximal Potential Inhibitory Effect (% MPIE) and (2) pain score. The % MPIEs was calculated by a series of steps, where the first is to sum the length of non-normal behaviours (behaviours 1,2,3) of each animal. A single value for the vehicle group was obtained by averaging all scores within the vehicle treatment group. The following calculation yields the MPIE value for each animal:
MPIE (%)=100−[(treatment sum/average vehicle value)×100% ]

The pain score is calculated from a weighted scale as described above. The duration of the behaviour is multiplied by the weight (rating of the severity of the response), and divided by the total length of observation to determine a pain rating for each animal. The calculation is represented by the following formula:
Pain rating=[0(To)+1(T1)+2(T2)+3(T3)]/(To+T1+T2+T3)

Compounds of the present invention were shown to be efficacious within a range of 30 mg/Kg and 0.1 mg/Kg.

CFA Induced Chronic Inflammatory Pain

Following a full week of acclimatization to the vivarium facility, 150 μL of the “Complete Freund's Adjuvant” (CFA) emulsion (CFA suspended in an oil/saline (1:1) emulsion at a concentration of 0.5 mg/mL) was injected subcutaneously into the plantar surface of the left hind paw of rats under light isoflurane anaesthesia. Animals were allowed to recover from the anaesthesia and the baseline thermal and mechanical nociceptive thresholds of all animals are assessed one week after the administration of CFA. All animals were habituated to the experimental equipment for 20 minutes on the day prior to the start of the experiment. The test and control articles were administrated to the animals, and the nociceptive thresholds measured at defined time points after drug administration to determine the analgesic responses to each of the six available treatments. The time points used were previously determined to show the highest analgesic effect for each test compound.

Thermal nociceptive thresholds of the animals were assessed using the Hargreaves test. Animals were placed in a Plexiglas enclosure set on top of an elevated glass platform with heating units. The glass platform is thermostatically controlled at a temperature of approximately 30° C. for all test trials. Animals were allowed to accommodate for 20 minutes following placement into the enclosure until all exploration behaviour ceases. The Model 226 Plantar/Tail Stimulator Analgesia Meter (IITC, Woodland Hills, Calif.) was used to apply a radiant heat beam from underneath the glass platform to the plantar surface of the hind paws. During all test trials, the idle intensity and active intensity of the heat source were set at 1 and 45 respectively, and a cut off time of 20 seconds was employed to prevent tissue damage.

The response thresholds of animals to tactile stimuli were measured using the Model 2290 Electrovonfrey anesthesiometer (IITC Life Science, Woodland Hills, Calif.) following the Hargreaves test. Animals were placed in an elevated Plexiglas enclosure set on a mire mesh surface. After 10 minutes of accommodation, pre-calibrated Von Frey hairs were applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force was used because it represent approximately 10% of the animals' body weight and it serves to prevent raising of the entire limb due to the use of stiffer hairs, which would change the nature of the stimulus. The compounds of the present invention were shown to be efficacious within a range of 30 mg/Kg and 0.1 mg/Kg.

Postoperative Models of Nociception

In this model, the hypealgesia caused by an intra-planar incision in the paw is measured by applying increased tactile stimuli to the paw until the animal withdraws its paw from the applied stimuli. While animals were anaesthetized under 3.5% isofluorane, which was delivered via a nose cone, a 1 cm longitudinal incision was made using a number 10 scalpel blade in the plantar aspect of the left hind paw through the skin and fascia, starting 0.5 cm from the proximal edge of the heel and extending towards the toes. Following the incision, the skin was apposed using 2, 3-0 sterilized silk sutures. The injured site was covered with Polysporin and Betadine. Animals were returned to their home cage for overnight recovery.

The withdrawal thresholds of animals to tactile stimuli for both operated (ipsilateral) and unoperated (contralateral) paws can be measured using the Model 2290 Electrovonfrey anesthesiometer (IITC Life Science, Woodland Hills, Calif.). Animals were placed in an elevated Plexiglas enclosure set on a mire mesh surface. After at least 10 minutes of acclimatization, pre-calibrated Von Frey hairs were applied perpendicularly to the plantar surface of both paws of the animals in an ascending order starting from the 10 g hair, with sufficient force to cause slight buckling of the hair against the paw. Testing continued until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

The compounds of the present invention were shown to be efficacious within a range of 30 mg/Kg and 0.1 mg/Kg.

Neuropathic Pain Model; Chronic Constriction Injury

Briefly, an approximately 3 cm incision was made through the skin and the fascia at the mid thigh level of the animals' left hind leg using a no. 10 scalpel blade. The left sciatic nerve was exposed via blunt dissection through the biceps femoris with care to minimize haemorrhagia. Four loose ligatures were tied along the sciatic nerve using 4-0 non-degradable sterilized silk sutures at intervals of 1 to 2 mm apart. The tension of the loose ligatures was tight enough to induce slight constriction of the sciatic nerve when viewed under a dissection microscope at a magnification of 4 fold. In the sham-operated animal, the left sciatic nerve was exposed without further manipulation. Antibacterial ointment was applied directly into the wound, and the muscle was closed using sterilized sutures. Betadine was applied onto the muscle and its surroundings, followed by skin closure with surgical clips.

The response thresholds of animals to tactile stimuli were measured using the Model 2290 Electrovonfrey anesthesiometer (IITC Life Science, Woodland Hills, Calif.). Animals were placed in an elevated Plexiglas enclosure set on a mire mesh surface. After 10 minutes of accommodation, pre-calibrated Von Frey hairs were applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represents approximately 10% of the animals' body weight and it serves to prevent raising of the entire limb due to the use of stiffer hairs, which would change the nature of the stimulus. The compounds of the present invention were shown to be efficacious within a range of 30 mg/Kg and 0.1 mg/Kg.

Thermal nociceptive thresholds of the animals were assessed using the Hargreaves test. Following the measurement of tactile thresholds, animals were placed in a Plexiglass enclosure set on top of an elevated glass platform with heating units. The glass platform is thermostatically controlled at a temperature of approximately 24 to 26° C. for all test trials. Animals were allowed to accommodate for 10 minutes following placement into the enclosure until all exploration behaviour ceases. The Model 226 Plantar/Tail Stimulator Analgesia Meter (IITC, Woodland Hills, Calif.) was used to apply a radiant heat beam from underneath the glass platform to the plantar surface of the hind paws. During all test trials, the idle intensity and active intensity of the heat source were set at 1 and 55 respectively, and a cut off time of 20 seconds was used to prevent tissue damage.

### BIOLOGICAL EXAMPLE 4

### Aconitine Induced Arrhythmia Test

The antiarrhythmic activity of compounds of the invention is demonstrated by the following test. Arrhythmia was provoked by intravenous administration of aconitine(2.0 μg/Kg) dissolved in physiological saline solution. Test drugs were intravenously administered 5 minutes after the administration of aconitine. Evaluation of the anti-arrhythmic activity was conducted by measuring the time from the aconitine administration to the occurrence of extrasystole (ES) and the time from the aconitine administration to the occurrence of ventricular tachycardia (VT).

In rats under isoflurane anaesthesia (¼ to ⅓ of 2%), a tracheotomy was performed by first creating an incision in the neck area, then isolating the trachea and making a 2 mm incision to insert tracheal tube 2 cm into the trachea such that the opening of the tube was positioned just on top of the mouth. The tubing was secured with sutures and attached to a ventilator for the duration of the experiment.

Incisions (2.5 cm) were then made into the femoral areas and using a blunt dissection probe, the femoral vessels were isolated. Both femoral veins were cannulated, one for pentobarbital anaesthetic maintenance (0.02-0.05 mL) and one for the infusion and injection of drug and vehicle. The femoral artery was cannulated with the blood pressure gel catheter of the transmitter.

The ECG leads were attached to the thoracic muscle in the Lead II position (upper right/above heart—white lead and lower left/below heart—red lead). The leads were secured with sutures.

All surgical areas were covered with gauze moistened with 0.9% saline. Saline (1-1.5 mL of a 0.9% solution) was supplied to moisten the areas post-surgery. The animals' ECG and ventillation were allowed to equilibrate for at least 30 minutes.

The arrhythmia was induced with a 2 μg/Kg/min aconitine infusion for 5 minutes. During this time the ECG was recorded and continuously monitoired. An intravenous bolus injection of test compound (10, 30 or 100 μg/Kg) resulted in a complete return to normal baseline ECG.

### BIOLOGICAL EXAMPLE 5

### Ischemia Induced Arrhythmia Test

Rodent models of ventricular arrhythmias, in both acute cardioversion and prevention paradigms have been employed in testing potential therapeutics for both atrial and ventricular arrhythmias in humans. Cardiac ischemia leading to myocardial infarction is a common cause of morbidity and mortality. The ability of a compound to prevent ischemia-induced ventricular tachycardia and fibrillation is an accepted model for determining the efficacy of a compound in a clinical setting for both atrial and ventricular tachycardia and fibrillation.

Anaesthesia is first induced by pentobarbital (i.p.), and maintained by an i.v. bolus infusion. Male SD rats have their trachea cannulated for artificial ventilation with room air at a stroke volume of 10 mL/Kg, 60 strokes/minute. The right femoral artery and vein are cannulated with PE50 tubing for mean arterial blood pressure (MAP) recording and intravenous administration of compounds, respectively.

The chest was opened between the 4^{th} and 5^{th} ribs to create a 1.5 cm opening such that the heart was visible. Each rat was placed on a notched platform and metal restraints were hooked onto the rib cage opening the chest cavity. A suture needle was used to penetrate the ventricle just under the lifted atrium and exited the ventricle in a downward diagonal direction so that a >30% to <50% occlusion zone (OZ) would be obtained. The exit position was ˜0.5 cm below where the aorta connects to the left ventricle. The suture was tightened such that a loose loop (occluder) was formed around a branch of the artery. The chest was then closed with the end of the occluder accessible outside of the chest.

Electrodes were placed in the Lead II position (right atrium to apex) for ECG measurement as follows: one electrode inserted into the right forepaw and the other electrode inserted into the left hind paw.

The body temperature, MAP, ECG, and heart rate were constantly recorded throughout the experiment. Once the critical parameters had stabilized, a 1-2 minute recording was taken to establish the baseline values. Infusion of the compound or contriol substances was initiated once baseline values were established. After a 5-minute infusion of compound or control, the suture was pulled tight to ligate the LCA and create ischemia in the left ventricle. The critical parameters were recorded continuously for 20 minutes after ligation, unless the MAP reached the critical level of 20-30 mmHg for at least 3 minutes, in which case the recording was stopped because the animal would be declared deceased and was then sacrificed. The ability of the compound to prevent arrhythmias and sustain near-normal MAP and HR was scored and compared to control.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A compound of formula (I): 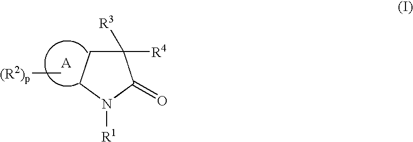
wherein:
p is 0, 1, 2, 3 or 4; 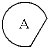
is a fused heteroaryl ring or a fused heterocyclyl ring;
R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;
or R¹ is aralkyl substituted by —C(O)N(R⁷)R⁸ where:
R⁷ is hydrogen, alkyl, aryl or aralkyl; and
R⁸ is hydrogen, alkyl, haloalkyl, —R¹⁰—CN, —R¹⁰—OR⁶, —R¹⁰—N(R⁵)R⁶ aryl aralkyl cycloalkyl, cycloalkylalkyl, heterocyclyl, heterocyclylalkyl, heteroaryl, heteroarylalkyl;
or R⁷ and R⁸, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;
or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;
or R¹ is —R¹⁰—N(R¹¹)R¹², —R¹⁰—N(R¹³)C(O)R¹² or —R¹⁰—N(R¹¹)C(O)N(R¹¹)R¹² where:
each R¹¹ is hydrogen, alkyl, aryl or aralkyl;
each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;
R¹³ is hydrogen, alkyl, aryl, arakyl or —C(O)R⁶;
and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;
or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;
each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵, —C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(═N—CN)N(R⁵)R⁶,
wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;
R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,
wherein X is bromo or chloro, each m is independently 0, 1, or 2 and each n is independently 1 or 2; and
wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain;
as a stereoisomer, enantiomer, tautomer thereof or mixtures thereof;
or a pharmaceutically acceptable salt, solvate or prodrug thereof.

2. The compound of claim 1 wherein:
p is 0, 1, 2, 3 or 4; 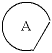
is a fused heteroaryl ring selected from the group consisting of pyrrolyl, pyrazolyl, pyridinyl, pyrimidinyl, thienyl and pyrazinyl;
R¹ is —R⁹—C(O)R⁶, —R⁹—C(O)OR⁶, —R⁹—OR⁶, —R⁹—CN, —R¹⁰—P(O)(OR⁶)₂, —R¹⁰—O—R¹⁰—OR⁶, hydrogen, alkyl, haloalkyl, cycloalkylalkyl, heterocyclylalkyl, aryl (optionally substituted by one or more substituents selected from the group consisting of halo and —R⁹—C(O)OR⁶), aralkyl (optionally substituted by one or more substituents selected from the group consisting of halo, haloalkyl, heteroaryl, —R⁹—OR⁶and —R⁹—C(O)OR⁶), heteroaryl (optionally substituted by one or more substituents selected from the group consisting of alkyl, halo, haloalkyl and —R⁹—OR⁶), or heteroarylalkyl (optionally substituted by one or more substituents selected from the group consisting of alkyl, halo, haloalkyl and —R⁹—OR⁶);
each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;
—R⁹—C(O)OR⁶, —R⁹—C(O)N(R⁵)R⁶, —N(R⁶)C(O)R⁵,
wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;
or two adjacent R² groups, together with the fused heteroaryl ring atoms to which they are directly attached, may form a fused ring selected from cycloalkyl, aryl, heterocyclyl and heteroaryl, and the remaining R² groups, if present, are as described above;
R³ is independently selected from the group consisting of hydrogen, halo, haloalkyl, —R⁹—OR⁶, —R⁹—OC(O)R⁶, —R⁹—CN, —R⁹—N(R⁵)R⁶, —R⁹—C(O)R⁵, —R⁹—C(O)X, —R⁹—C(O)OR⁶ and —N(R⁶)C(O)OR⁶, wherein X is chloro or bromo;
R⁴ is independently selected from the group consisting of alkyl, aryl, aralkyl, aralkynyl, heteroaryl, heteroarylalkyl, —R⁹—C(O)R⁵, —N(R⁶)C(O)N(R⁵)R⁶, —R⁹—NO₂, —R⁹—N(R⁵)R⁶, —R⁹—C(O)OR⁶, —R⁹—N(R⁶)C(O)OR⁶ and —Si(R⁶)₃,
wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain.

3. The compound of claim 2 wherein:
p is 0, 1, 2, 3 or 4; 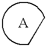
is a fused heteroaryl ring selected from the group consisting of pyrrolyl, pyrazolyl, pyridinyl, pyrimidinyl, thienyl and pyrazinyl;
R¹ is alkyl, aryl or aralkyl, where each of the aryl or aralkyl group for R¹ is optionally substituted by one or more substituents selected from the group consisting of halo, haloalkyl, heteroaryl, —R⁹—OR⁶ and —R⁹—C(O)OR⁶;
each R² is independently selected from the group consisting of alkyl, halo, aryl, heteroaryl and —R⁹—OR⁶,
wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
R³ is hydrogen, halo, —R⁹—OR⁶ or —R⁹—OC(O)R⁶;
R⁴ is independently selected from the group consisting of alkyl, aryl, aralkynyl, heteroaryl, heteroarylalkyl, —R⁹—C(O)R⁵, —N(R⁶)C(O)N(R⁵)R⁶, —R⁹—NO₂, —R⁹—N(R⁵)R⁶, —R⁹—C(O)OR⁶ and —Si(R⁶)₃,
wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

4. The compound of claim 3 wherein:
p is 0, 1, 2, 3 or 4; 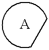
is a fused heteroaryl ring selected from the group consisting of pyrrolyl, pyrazolyl, pyridinyl, pyrimidinyl, thienyl and pyrazinyl;
R¹ is alkyl, aryl or aralkyl, where each of the aryl or aralkyl group for R¹ is optionally substituted by one or more substituents selected from the group consisting of halo, haloalkyl, heteroaryl, —R⁹—OR⁶ and —R⁹—C(O)OR⁶;
each R² is independently selected from the group consisting of alkyl, halo, aryl, heteroaryl and —R⁹—OR⁶,
wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
R³ is hydrogen, halo, —R⁹—OR⁶ or —R⁹—OC(O)R⁶;
R⁴ is —R⁹—C(O)R⁵;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

5. The compound of claim 4 selected from the group consisting of the following:
3-hydroxy-3-[2-oxo-2-(2-thienyl)ethyl]-1-pentyl-1,3-dihydro-2H-pyrrolo[2,3-b]pyridin-2-one; and
3-[2-(2-furyl)-2-oxoethyl]-3-hydroxy-1-pentyl-1,3-dihydro-2H-pyrrolo[2,3-b]pyridin-2-one.

6. The compound of claim 3 wherein:
p is 0, 1, 2, 3 or 4; 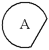
is a fused heteroaryl ring selected from the group consisting of pyrrolyl, pyrazolyl, pyridinyl, pyrimidinyl, thienyl and pyrazinyl;
R¹ is aralkyl (optionally substituted by one or more substituents selected from the group consisting of halo, haloalkyl, heteroaryl, —R⁹—OR⁶ and —R⁹—C(O)OR⁶);
each R² is independently selected from the group consisting of alkyl, halo, aryl, heteroaryl and —R⁹—OR⁶,
wherein each of the aryl and heteroaryl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;
R³ is hydrogen, halo, —R⁹—OR⁶ or —R⁹—OC(O)R⁶;
R⁴ is heterocyclylalkyl, heteroaryl or heteroarylalkyl, each optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

7. The compound of claim 6 wherein:
p is 0, 1, 2, 3 or 4; 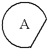
is a fused heteroaryl ring selected from the group consisting of pyrrolyl, pyrazolyl, pyridinyl, pyrimidinyl, thienyl and pyrazinyl;
R¹ is aralkyl (optionally substituted by one or more substituents selected from the group consisting of halo, haloalkyl, heteroaryl, —R⁹—OR⁶ and —R⁹—C(O)OR⁶);
each R² is each independently selected from the group consisting of alkyl, halo, phenyl, benzodioxolyl and —R⁹—OR⁶,
R³ is hydrogen, halo, —R⁹—OR⁶ or —R⁹—OC(O)R⁶;
R⁴ is heterocyclylalkyl, heteroaryl or heteroarylalkyl, each optionally substituted by one or more substituents selected from the group consisting of halo, heterocyclyl, and —R⁹—OR⁶;
each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and
each R⁹ is a direct bond or an optionally substituted straight or branched alkylene chain, an optionally substituted straight or branched alkenylene chain or an optionally substituted straight or branched alkynylene chain.

8. The compound of claim 3 wherein:
p is 0; 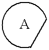
is a fused heteroaryl ring selected from the group consisting of pyrrolyl, pyrazolyl, pyridinyl, pyrimidinyl, thienyl and pyrazinyl;
R¹ is aralkyl (optionally substituted by one or more substituents selected from the group consisting of halo, haloalkyl, heteroaryl, —R⁹—OR⁶ and —R⁹—C(O)OR⁶);
R³ is —R⁹—OR⁶;
R⁴ is aryl, aralkyl or aralkynyl,
wherein each of the aryl, aralkyl and aralkynyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO², —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

9. The compound of claim 8 wherein:
p is 0; 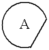
is a fused heteroaryl ring selected from the group consisting of pyrrolyl, pyrazolyl, pyridinyl, pyrimidinyl, thienyl and pyrazinyl;
R¹ is aralkyl (optionally substituted by one or more substituents selected from the group consisting of halo, haloalkyl, heteroaryl, —R⁹—OR⁶ and —R⁹—C(O)OR⁶);
R³ is R⁹—OR⁶;
R⁴ is aryl, aralkyl or aralkynyl,
wherein each of the aryl, aralkyl and aralkynyl groups for R⁴ is optionally substituted by one or more substituents selected from the group consisting of halo, oxo and —R⁹—OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain.

10. The compound of claim 2 wherein:
p is 0, 1, 2, 3 or 4; 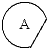
is a fused heteroaryl ring selected from the group consisting of pyrrolyl, pyrazolyl, pyridinyl, pyrimidinyl, thienyl and pyrazinyl;
R¹ is hydrogen, alkyl, haloalkyl or cycloalkylalkyl;
each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;
—R⁹—C(O)OR⁶, —R⁹—C(O)N(R⁵)R⁶, —N(R⁶)C(O)R⁵,
wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the heteroaryl ring atoms to which they are directly attached, may form a fused ring selected from cycloalkyl, aryl, heterocyclyl and heteroaryl, and the remaining R² groups, if present, are as described above;
R³is hydrogen, halo or —R⁹—OR⁶;
R⁴ is independently selected from the group consisting of alkyl, aryl, aralkynyl, heteroaryl, heteroarylalkyl, —R⁹—C(O)R⁵, —R⁹—N(R⁶)C(O)OR⁶, —N(R⁶)C(O)N(R⁵)R⁶, —R⁹—NO₂, —R⁹—N(R⁵)R⁶, —R⁹—C(O)OR⁶, and —Si(R⁶)₃,
wherein each of the aryl, aralkynyl, heteroaryl and heteroarylalkyl groups for R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

11. The compound of claim 10 wherein:
p is 0, 1, 2, 3 or 4; 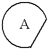
is a fused heteroaryl ring selected from the group consisting of pyrrolyl, pyrazolyl, pyridinyl, pyrimidinyl, thienyl and pyrazinyl
R¹ is hydrogen, alkyl, haloalkyl or cycloalkylalkyl;
each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;
—R⁹—C(O)OR⁶, —R⁹—C(O)N(R⁵)R⁶, —N(R⁶)C(O)R⁵,
wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the heteroaryl ring atoms to which they are directly attached, may form a fused ring selected from cycloalkyl, aryl, heterocyclyl and heteroaryl, and the remaining R² groups, if present, are as described above;
R³ is hydrogen or —R⁹—OR⁶;
R⁴ is heteroaryl optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

12. The compound of claim 11 wherein:
p is 0, 1, 2, 3 or 4; 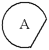
is a fused heteroaryl ring selected from the group consisting of pyrrolyl, pyrazolyl, pyridinyl, pyrimidinyl, thienyl and pyrazinyl;
R¹ is alkyl;
each R² is independently selected from the group consisting of alkyl, halo, haloalkyl and —R⁹—OR⁶;
R³ is hydrogen or —R⁹—OR⁶;
R⁴ is heteroaryl optionally substituted by one or more substituents selected from the group consisting of halo, —R⁹—OR⁶ and —N(R⁶)C(O)R⁵;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

13. The compound of claim 12 wherein:
p is 0, 1, 2, 3 or 4; 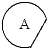
is a fused heteroaryl ring selected from the group consisting of pyrrolyl, pyrazolyl, pyridinyl, pyrimidinyl, thienyl and pyrazinyl;
R¹ is alkyl;
each R² is independently selected from the group consisting of alkyl, halo, haloalkyl and —R⁹—OR⁶;
R³ is hydrogen or —R⁹—OR⁶;
R⁴ is benzodioxolyl optionally substituted by one or more substituents selected from the group consisting of halo and —R⁹—OR⁶;
each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and
each R⁹ is a direct bond or an optionally substituted straight or branched alkylene chain, an optionally substituted straight or branched alkenylene chain or an optionally substituted straight or branched alkynylene chain.

14. The compound of claim 13 selected from the group consisting of the following:
3-(1,3-benzodioxol-5-yl)-3-hydroxy-1-pentyl-1H-pyrrolo[1,2-b]pyrazol-2(3H)-one;
4-(1,3-benzodioxol-5-yl)-4-hydroxy-6-pentyl-4,6-dihydro-5H-thieno[2,3-b]pyrrol-5-one;
6-(1,3-benzodioxol-5-yl)-6-hydroxy-4-pentyl-4,6-dihydro-5H-thieno[3,2-b]pyrrol-5-one;
3-hydroxy-3-(6-hydroxy-1,3-benzodioxol-5-yl)-1-pentyl-1,3-dihydro-2H-pyrrolo[2,3-b]pyridin-2-one;
3-hydroxy-3-(6-hydroxy-1,3-benzodioxol-5-yl)-1-pentyl-1,3-dihydro-2H-pyrrolo[3,2-b]pyridin-2-one;
3-hydroxy-3-(6-hydroxy-1,3-benzodioxol-5-yl)-1-pentyl-1,3-dihydro-2H-pyrrolo[3,2-c]pyridin-2-one;
6-hydroxy-6-(6-hydroxy-1,3-benzodioxol-5-yl)-4-pentyl-4,6-dihydro-5H-thieno[3,2-b]pyrrol-5-one;
3-(6-hydroxy-1,3-benzodioxol-5-yl)-1-pentyl-1,3-dihydro-2H-pyrrolo[2,3-b]pyridin-2-one;
3-(6-hydroxy-1,3-benzodioxol-5-yl)-1-pentyl-1,3-dihydro-2H-pyrrolo[3,2-b]pyridin-2-one;
3-(6-hydroxy-1,3-benzodioxol-5-yl)-1-pentyl-1,3-dihydro-2H-pyrrolo[3,2-c]pyridin-2-one;
6-(6-hydroxy-1,3-benzodioxol-5-yl)-4-pentyl-4,6-dihydro-5H-thieno[3,2-b]pyrrol-5-one;
3-(6-hydroxy-1,3-benzodioxol-5-yl)-3-(hydroxymethyl)-1-pentyl-1,3-dihydro-2H-pyrrolo[2,3-b]pyridin-2-one;
3-(6-hydroxy-1,3-benzodioxol-5-yl)-3-(hydroxymethyl)-1-pentyl-1,3-dihydro-2H-pyrrolo[3,2-b]pyridin-2-one;
3-(6-hydroxy-1,3-benzodioxol-5-yl)-3-(hydroxymethyl)-1-pentyl-1,3-dihydro-2H-pyrrolo[3,2-c]pyridin-2-one;
6-(6-hydroxy-1,3-benzodioxol-5-yl)-6-(hydroxymethyl)-4-pentyl-4,6-dihydro-5H-thieno[3,2-b]pyrrol-5-one;
3-(1,3-benzodioxol-5-yl)-3-hydroxy-1-pentyl-1,3-dihydro-2H-pyrrolo[2,3-b]pyridin-2-one; and
3-(1,3-benzodioxol-5-yl)-1-pentyl-1,3-dihydro-2H-pyrrolo[2,3-b]pyridin-2-one.

15. The compound of claim 10 wherein:
p is 0, 1, 2, 3 or 4; 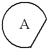
is a fused heteroaryl ring selected from the group consisting of pyrrolyl, pyrazolyl, pyridinyl, pyrimidinyl, thienyl and pyrazinyl;
R¹ is alkyl;
each R² is independently selected from the group consisting of alkyl, halo, haloalkyl and —R⁹—OR⁶;
R³ is hydrogen, halo or —R⁹—OR⁶;
R⁴ is independently selected from the group consisting of —R⁹—C(O)R⁵ and —R⁹—N(R⁶)C(O)OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and
each R⁹ is a direct bond or an optionally substituted straight or branched alkylene chain, an optionally substituted straight or branched alkenylene chain or an optionally substituted straight or branched alkynylene chain.

16. The compound of claim 2 wherein:
p is 0; 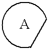
is a fused heteroaryl ring selected from the group consisting of pyrrolyl, pyrazolyl, pyridinyl, pyrimidinyl, thienyl and pyrazinyl;
R¹ is alkyl or aralkyl (optionally substituted by one or more substituents selected from the group consisting of halo, haloalkyl, —R⁹—OR⁶, heteroaryl and —R⁹—C(O)OR⁶);
R³ is —R⁹—C(O)X, —R⁹—C(O)OR⁶ and —R⁹—C(O)N(R⁵)R⁶ where X is bromo or chloro;
R⁴ is independently selected from the group consisting of —R⁹—C(O)R⁵ and heteroaryl optionally substituted by one or more substituents selected from the group consisting of halo and R⁹—OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

17. The compound of claim 2 wherein:
p is 0, 1, 2, 3 or 4; 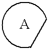
is a fused heteroaryl ring selected from the group consisting of pyrrolyl, pyrazolyl, pyridinyl, pyrimidinyl, thienyl and pyrazinyl;
R¹ is alkyl or aralkyl optionally substituted by one or more substituents selected from the group consisting of halo and —R⁹—C(O)OR⁶;
each R² is independently selected from the group consisting of alkyl, halo, haloalkyl, haloalkenyl, cycloalkyl, cycloalkylalkyl, aryl, aralkyl, heterocyclyl, heterocyclylalkyl, heteroaryl, heteroarylalkyl, —R⁹—OR⁶, —R⁹—N(R⁵)R⁶, —R⁹—C(O)R⁵;
—R⁹—C(O)OR⁶, —R⁹—C(O)N(R⁵)R⁶, —N(R⁶)C(O)R⁵,
wherein each of the cycloalkyl, cycloalkylalkyl, aryl, aralkyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵ and —N(R⁶)S(O)ₙR⁵, wherein each m is independently 0, 1, or 2 and each n is independently 1 or 2;
or two adjacent R² groups, together with the heteroaryl ring atoms to which they are directly attached, may form a fused ring selected from cycloalkyl, aryl, heterocyclyl and heteroaryl, and the remaining R² groups, if present, are as described above;
R³ and R⁴ together form ═NS(O)₂R⁶, ═N—R¹⁵, ═N—O—R⁶ or ═R^{9a}—C(O)R⁶,
where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

18. The compound of claim 17 wherein:
p is 0, 1, 2, 3 or 4; 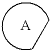
is a fused heteroaryl ring selected from the group consisting of pyrrolyl, pyrazolyl, pyridinyl, pyrimidinyl, thienyl and pyrazinyl;
R¹ is alkyl or aralkyl optionally substituted by one or more substituents selected from the group consisting of halo and —R⁹—C(O)OR⁶;
each R² is independently selected from the group consisting of alkyl, halo and haloalkyl;
or two adjacent R² groups, together with the heteroaryl ring atoms to which they are directly attached, may form a fused ring selected from cycloalkyl, aryl, heterocyclyl and heteroaryl, and the remaining R² groups, if present, are as described above;
R³ and R⁴ together form ═NS(O)₂R⁶, ═N—R¹⁵, ═N—O—R⁶ or ═R^{9a}—C(O)R⁶,
where R^{9a} is a straight or branched alkenylene chain wherein the alkenylene chain is attached to the carbon to which R³ and R⁴ is attached through a double bond and R¹⁵ is a N-heterocyclyl optionally substituted by alkyl, haloalkyl or —R⁹—OR⁶;
each R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl; and
each R⁹ is a direct bond or an optionally substituted straight or branched alkylene chain, an optionally substituted straight or branched alkenylene chain or an optionally substituted straight or branched alkynylene chain.

19. The compound of claim 2 wherein:
p is 0, 1, 2, 3 or 4; 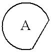
is a fused heteroaryl ring selected from the group consisting of pyridinyl, pyrimidinyl, thienyl and pyrazinyl;
R¹ is alkyl;
each R² is independently selected from the group consisting of alkyl, halo, haloalkyl and —R⁹OR⁶;
R³ is independently selected from the group consisting of halo, —R⁹—CN, —R⁹—N(R⁵)R⁶ and —N(R⁶)C(O)OR⁶;
R⁴ is heteroaryl optionally substituted by one or more substituents selected from the group consisting of alkyl, halo, haloalkyl, and —R⁹—OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

20. The compound of claim 19 wherein:
p is 0, 1, 2, 3 or 4; 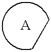
is a fused heteroaryl ring selected from the group consisting of pyridinyl, pyrimidinyl, thienyl and pyrazinyl;
R¹ is alkyl;
each R² is independently selected from the group consisting of alkyl, halo, haloalkyl and —R⁹—OR⁶;
R³ is independently selected from the group consisting of halo, —R⁹—CN, —R⁹—N(R⁵)R⁶ and —N(R⁶)C(O)OR⁶;
R⁴ is benzodioxolyl optionally substituted by one or more substituents selected from the group consisting of alkyl, halo, haloalkyl, and —R⁹—OR⁶;
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl; and
each R⁹ is a direct bond or an optionally substituted straight or branched alkylene chain, an optionally substituted straight or branched alkenylene chain or an optionally substituted straight or branched alkynylene chain.

21. The compound of claim 20 selected from the group consisting of the following:
3-(1,3-benzodioxol-5-yl)-3-fluoro-1-pentyl-1,3-dihydro-2H-pyrrolo[2,3-b]pyridin-2-one;
3-(1,3-benzodioxol-5-yl)-2-oxo-1-pentyl-2,3-dihydro-1H-pyrrolo[2,3-b]pyridine-3-carbonitrile; and
3-(1,3-benzodioxol-5-yl)-3-(benzylamino)-1-pentyl-1,3-dihydro-2H-pyrrolo[2,3-b]pyridin-2-one

22. A method of treating, preventing or ameliorating a disease or a condition of a mammal selected from the group consisting of pain, depression, cardiovascular diseases, respiratory diseases, and psychiatric diseases, and combinations thereof, wherein the method comprises administering to the mammal in need thereof a therapeutically effective amount of a compound of formula (I): 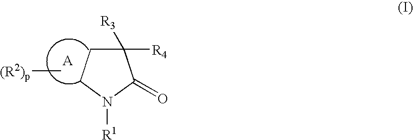
wherein:
p is 0, 1, 2, 3 or 4; 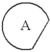
is a fused heteroaryl ring or a fused heterocyclyl ring;
R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;
or R¹ is aralkyl substituted by —C(O)N(R⁷)R⁸ where:
R⁷ is hydrogen, alkyl, aryl or aralkyl; and
R⁸ is hydrogen, alkyl, haloalkyl, —R¹⁰—CN, —R¹⁰—OR⁶, —R¹⁰—N(R⁵)R⁶, aryl, aralkyl, cycloalkyl, cycloalkylalkyl, heterocyclyl, heterocyclylalkyl, heteroaryl, heteroarylalkyl;
or R⁷ and R⁸, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;
or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;
or R¹ is —R¹⁰—N(R¹¹)R¹², —R¹⁰—N(R¹³)C(O)R¹² or —R¹⁰—N(R¹¹)C(O)N(R¹¹)R¹² where:
each R¹¹ is hydrogen, alkyl, aryl or aralkyl;
each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;
R¹³ is hydrogen, alkyl, aryl, arakyl or —C(O)R⁶;
and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;
or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;
each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵, —C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N( R⁵)R⁶, —N(R⁶)S(O)ₙR⁵, —N(R⁶)S(O)ₙN(R⁵)R⁶, —R⁹—S(O)ₙN(R⁵)R⁶—N(R⁶)C(═NR⁶)N(R⁵)R⁶, and —N(R⁶)C(═N—CN)N(R⁵)R⁶,
wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;
R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,
wherein X is bromo or chloro, each m is independently 0, 1, or 2 and each n is independently 1 or 2; and
wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain;
as a stereoisomer, enantiomer, tautomer thereof or mixtures thereof;
or a pharmaceutically acceptable salt, solvate or prodrug thereof.

23. A method of claim 22, wherein said disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, peripheral nerve injury, pain associated with narcotic drug addiction withdrawal and combinations thereof.

24. A method of claim 22, wherein said disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schitzophrenia, sodium channel toxin related Illnesses, familial erythermalgia, primary erythermalgia, familial rectal pain, cancer, narcotic drug addiction, epilepsy, partial and general tonic seizures, restless leg syndrome, arrhythmias, fibromyalgia, neuroprotection under ischaemic conditions caused by stroke or neural trauma, tachy-arrhythmias, atrial fibrillation and ventricular fibrillation.

25. A method of treating pain through inhibition of ion flux through a voltage-dependent sodium channel in a mammal, wherein the method comprises administering to the mammal in need thereof a therapeutically effective amount of a compound of formula (I): 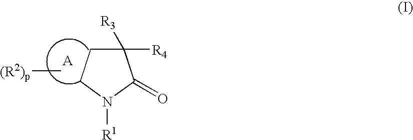
wherein:
p is 0, 1, 2, 3 or 4; 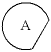
is a fused heteroaryl ring or a fused heterocyclyl ring;
R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;
or R¹ is aralkyl substituted by —C(O)N(R⁷)R⁸ where:
R⁷ is hydrogen, alkyl, aryl or aralkyl; and
R⁸ is hydrogen, alkyl, haloalkyl, —R¹⁰—CN, —R¹⁰—OR⁶, —R¹⁰—N(R⁵)R⁶, aryl, aralkyl, cycloalkyl, cycloalkylalkyl, heterocyclyl, heterocyclylalkyl, heteroaryl, heteroarylalkyl;
or R⁷ and R⁸, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;
or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;
or R¹ is —R¹⁰—N(R¹¹)R¹², —R¹⁰—N(R¹³)C(O)R¹² or —R¹⁰—N(R¹¹)C(O)N(R¹¹)R¹² where:
each R¹¹ is hydrogen, alkyl, aryl or aralkyl;
each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;
R¹³ is hydrogen, alkyl, aryl, arakyl or —C(O)R⁶;
and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;
or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;
each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵—C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁵)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(═N—CN)N(R⁵)R⁶,
wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;
R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,
wherein X is bromo or chloro, each m is independently 0, 1, or 2 and each n is independently 1 or 2; and
wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain;
as a stereoisomer, enantiomer, tautomer thereof or mixtures thereof;
or a pharmaceutically acceptable salt, solvate or prodrug thereof.

26. A method of decreasing ion flux through a voltage-dependent sodium channel in a cell in a mammal, wherein the method comprises contacting the cell with a compound of formula (I): 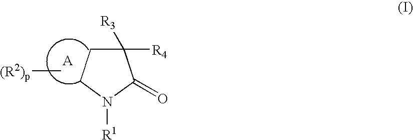
wherein:
p is 0, 1, 2, 3 or 4; 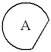
is a fused heteroaryl ring or a fused heterocyclyl ring;
R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;
or R¹ is aralkyl substituted by —C(O)N(R⁷)R⁸ where:
R⁷ is hydrogen, alkyl, aryl or aralkyl; and
R⁸ is hydrogen, alkyl, haloalkyl, —R¹⁰—CN, —R¹⁰—OR⁶, —R¹⁰—N(R⁵)R⁶ aryl, aralkyl, cycloalkyl, cycloalkylalkyl, heterocyclyl, heterocyclylalkyl, heteroaryl, heteroarylalkyl;
or R⁷ and R⁸, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;
or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;
or R¹ is —R¹⁰—N(R¹¹)R¹², —R¹⁰—N(R¹³)C(O)R¹² or —R¹⁰—N(R¹¹)C(O)N(R¹¹)R¹² where:
each R¹¹ is hydrogen, alkyl, aryl or aralkyl;
each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;
R¹³ is hydrogen, alkyl, aryl, arakyl or —C(O)R⁶;
and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;
or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;
each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵, —C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(═N—CN)N(R⁵)R⁶,
wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;
R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,
wherein X is bromo or chloro, each m is independently 0, 1, or 2 and each n is independently 1 or 2; and
wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴is attached through a double bond and R¹⁵ is a heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain;
as a stereoisomer, enantiomer, tautomer thereof or mixtures thereof;
or a pharmaceutically acceptable salt, solvate or prodrug thereof.

27. A pharmaceutical composition comprising a pharmaceutically acceptable excipient and a compound of formula (I): 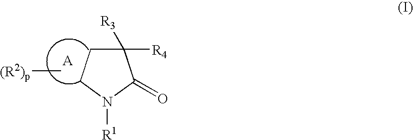
wherein:
p is 0, 1, 2, 3 or 4; 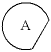
is a fused heteroaryl ring or a fused heterocyclyl ring;
R¹ is hydrogen, alkyl, alkenyl, alkynyl, haloalkyl, aryl, aralkyl, aralkenyl, cycloalkyl, cycloalkylalkyl, heteroaryl, heterocyclyl, —R⁹—C(O)R⁶, —R⁹—C(O)OR⁶, —R⁹—C(O)N(R⁵)R⁶, —R⁹—OR⁶, —R⁹—CN, —R¹⁰—P(O)(OR⁶)₂ or —R¹⁰—O—R¹⁰—OR⁶;
or R¹ is aralkyl substituted by —C(O)N(R⁷)R⁸ where:
R⁷ is hydrogen, alkyl, aryl or aralkyl; and
R⁸ is hydrogen, alkyl, haloalkyl, —R¹⁰—CN, —R¹⁰—OR⁶, —R¹⁰—N(R⁵)R⁶, aryl, aralkyl, cycloalkyl, cycloalkylalkyl, heterocyclyl, heterocyclylalkyl, heteroaryl, heteroarylalkyl;
or R⁷ and R⁸, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl group for R⁷ and R⁸ is optionally substituted by one or more substituents selected from the group consisting of alkyl, cycloalkyl, aryl, aralkyl, halo, haloalkyl, —R⁹—CN, —R⁹—OR⁶, heterocyclyl and heteroaryl;
or R¹ is aralkyl substituted by one or more substituents selected from the group consisting of —R⁹—OR⁶, —R⁹—C(O)OR⁶, halo, haloalkyl, alkyl, nitro, cyano, aryl (optionally substituted by cyano), aralkyl (optionally substituted by one or more alkyl groups), heterocyclyl and heteroaryl;
or R¹ is —R¹⁰—N(R¹¹)R¹², —R¹⁰—N(R¹³)C(O)R¹² or —R¹⁰—N(R¹¹)C(O)N(R¹¹)R¹² where:
each R¹¹ is hydrogen, alkyl, aryl or aralkyl;
each R¹² is hydrogen, alkyl, haloalkyl, cycloalkyl, cycloalkylalkyl, aryl, aralkyl, heterocyclyl, heterocyclylalkyl, heteroaryl, heteroarylalkyl, —R¹⁰—OC(O)R⁶, —R¹⁰—C(O)OR⁶, —R¹⁰—C(O)N(R⁵)R⁶, —R¹⁰—C(O)R⁶, —R¹⁰—OR⁶, or —R¹⁰—CN;
R¹³ is hydrogen, alkyl, aryl, arakyl or —C(O)R⁶;
and wherein each aryl, aralkyl, cycloalkyl, cycloalkylalkyl, heterocyclyl, heterocyclylalkyl, heteroaryl and heteroarylalkyl groups for R¹¹ and R¹² is optionally substituted by one or more substituents selected from the group consisting of alkyl, cycloalkyl, aryl, aralkyl, halo, haloalkyl, nitro, —R⁹—CN, —R⁹—OR⁶, —R⁹—C(O)R⁶, heterocyclyl and heteroaryl;
or R¹ is heterocyclylalkyl or heteroarylalkyl where the heterocyclylalkyl or the heteroaryl group is optionally substituted by one or more substituents selected from the group consisting of alkyl, halo, haloalkyl, —R⁹—OR⁶, —R⁹—C(O)OR⁶, aryl and aralkyl;
each R² is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —C(S)R⁵, —C(R⁵)₂C(O)R⁶, —R⁹—C(O)OR⁶, —C(S)OR⁵, —R⁹—C(O)N(R⁵)R⁶, —C(S)N(R⁵)R⁶, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(═N—CN)N(R⁵)R⁶,
wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R² is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or two adjacent R² groups, together with the fused heteroaryl ring or the fused heterocyclyl ring atoms to which they are directly attached, may form a fused ring selected from cycloalkyl, aryl, heterocyclyl and heteroaryl, and the remaining R² groups, if present, are as described above;
R³ and R⁴ are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, aralkynyl, heterocyclyl, heterocyclylalkyl, heteroaryl, heteroarylalkyl, —R⁹—CN, —R⁹—NO₂, —R⁹—OR⁶, —R⁹—N(R⁵)R⁶, —N═C(R⁵)R⁶, —S(O)ₘR⁵, —R⁹—C(O)R⁵; —R⁹—C(O)X, —C(S)R⁵, —C(R⁵)₂C(O)R⁶, —R⁹—OC(O)R⁶, —R⁹—C(O)OR⁶, —C(S)OR⁵, —R⁹—C(O)N(R⁵)R⁶, —C(S)N(R⁵)R⁶, —Si(R⁶)₃, —N(R⁶)C(O)R⁵, —N(R⁶)C(S)R⁵, —N(R⁶)C(O)OR⁶, —N(R⁶)C(S)OR⁵, —N(R⁶)C(O)N(R⁵)R⁶, —N(R⁶)C(S)N(R⁵)R⁶, —N(R⁶)S(O)ₙR⁵, —N(R⁶)S(O)ₙN(R⁵)R⁶, —R⁹—S(O)ₙN(R⁵)R⁶, —N(R⁶)C(═NR⁶)N(R⁵)R⁶, and —N(R⁶)C(N═C(R⁵)R⁶)N(R⁵)R⁶,
wherein X is bromo or chloro, each m is independently 0, 1, or 2 and each n is independently 1 or 2; and
wherein each of the cycloalkyl, cycloalkylalkyl, aryl, aralkyl, aralkenyl, aralkynyl, heterocyclyl, heterocyclylalkyl, heteroaryl, and heteroarylalkyl groups for R³ and R⁴ is optionally substituted by one or more substituents selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁹—CN, —R⁹—NO₂, —R⁹—OR⁶, —R⁹—N(R⁵)R⁶, —S(O)ₘR⁵, —R⁹—C(O)R⁵; —R⁹—C(O)OR⁶, —R⁹—C(O)N(R⁵)R⁶, —N(R⁶)C(O)R⁵, and —N(R⁶)S(O)ₙR⁵, wherein each m is independently 0, 1, or 2 and each n is independently 1 or 2;
or R³ and R⁴ together may form ═NS(O)₂R⁶, ═N—R¹⁵, ═N—O—R⁶ or ═R^{9a}—C(O)R⁶ (where R^{9a} is a straight or branched alkenylene chain wherein the alkenylene chain is attached to the carbon to which R³ and R⁴ is attached through a double bond and R¹⁵ is a heterocyclyl optionally substituted by alkyl, haloalkyl or —R⁹—OR⁶);
each R⁵ and R⁶ is independently selected from group consisting of hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl and optionally substituted heteroaryl;
or when R⁵ and R⁶ are each attached to the same nitrogen atom, then R⁵ and R⁶, together with the nitrogen atom to which they are attached, may form a N-heterocyclyl or N-heteroaryl;
each R⁹ is a direct bond or an optionally substituted straight or branched alkylene chain, an optionally substituted straight or branched alkenylene chain or an optionally substituted straight or branched alkynylene chain; and
each R¹⁰ is an optionally substituted straight or branched alkylene chain, an optionally substituted straight or branched alkenylene chain or an optionally substituted straight or branched alkynylene chain;
as a stereoisomer, enantiomer, tautomer thereof or mixtures thereof;
or a pharmaceutically acceptable salt, solvate or prodrug thereof.
